(19) United States
(12) Patent Application Publication
(10) Pub. No.: US 2010/0239652 A1
(43) Pub. Date: Sep. 23, 2010

(54) IMMUNOLIPOSOMES FOR TREATMENT OF CANCER

(75) Inventors: Christoph Rochlitz, Riehen (CH); Christoph Mamot, Basel (CH)

Correspondence Address: DICKSTEIN SHAPIRO LLP, 1633 Broadway, NEW YORK, NY, 10019, US

(73) Assignee: UNIVERSITATSSPITAL BASEL, BASEL (CH)

(21) Appl. No.: 12/680,698

(22) PCT Filed: Sep. 26, 2008
(86) PCT No.: PCT/EP08/62958

§ 371 Date: Mar. 29, 2010

(51) Int. Cl. A61K 9/127; A61K 39/395; A61P 35/00

(52) U.S. Cl. 424/450; 424/174.1

(57) ABSTRACT

The present invention relates to immunoliposomes for multiple treatment of human patients suffering from cancer, particularly a cancer represented by a locally advanced or metastatic tumor and to compositions used in said method. The invention further relates to the use of immunoliposomes for the treatment of multi-drug resistance in cancer therapy.

The present invention is in the area of cancer treatment. In particular, the invention relates to first- and higher-line treatment of human patients suffering from cancer, particularly a cancer represented by a locally advanced or metastatic tumor and to compositions used in said method.

The epidermal growth factor receptor (EGFR) is a tyrosine kinase receptor of the ErbB family that is abnormally activated in many epithelial tumors. Receptor activation leads to recruitment and phosphorylation of several downstream intracellular substrates, leading to mitogenic signaling and other tumor-promoting cellular activities. In human tumors, receptor overexpression correlates with a more aggressive clinical course (1, 2). Monoclonal antibodies directed at the ligand-binding extracellular domain and low-molecular weight inhibitors of the receptor's tyrosine kinase are currently in advanced stages of clinical development.

Among available anti-EGFR MAbs, the one best characterized is the chimeric human:murine MAb cetuximab. Cetuximab is a potent inhibitor of the growth of cultured cancer cells that have an active autocrine EGFR loop. A series of phase I, phase II and phase III studies of cetuximab given alone or in combination either with chemotherapy or radiation have now been completed. Cetuximab was found to be safe but showed some side effects including an acneiform skin rash in up to 40-70% of all treated patients and anaphylactoid or anaphylactic reactions that occurred in 2% of patients. Nonneutralizing human antibodies against chimeric antibodies were detected in 4% of patients. The optimal biologic dose, as determined by saturation of antibody clearance, was found to be in the range of 200 to 400 mg/m2 per week (3). Cetuximab is now considered part of standard therapy in patients with colorectal cancer and in head&neck tumors in many countries.

Doxorubicin is one of the most widely used anticancer drugs for the treatment of solid tumors and hematologic malignancies. It is active against a variety of cancer types, and is used extensively as a single agent and in combination chemotherapy regimens. In addition to its pivotal role in the treatment of breast cancer, doxorubicin has also demonstrated antitumor activity in ovarian, cervical, endometrial, gastric, bladder, and small-cell lung cancer, uterine sarcoma, acute lymphoblastic leukemia, Hodgkin's and non-Hodgkin's lymphoma, multiple myeloma, and soft tissue and bone sarcomas. While doxorubicin displays an excellent antitumor activity profile, its use in clinical practice is limited by drug-associated toxicities, particularly myelo suppression and cardiotoxicity (citation: “Principals and Practice of Oncology, DeVita, 6^{th} edition”).

Liposomal encapsulation of doxorubicin was used to alter the tissue distribution and pharmacokinetics of the drug and to increase its therapeutic index. Pegylated liposomal doxorubicin (DOXYL, Ortho Biotech Products LP, Bridgewater, N.J.; CAELYX, Schering Plough, Kenilworth, N.J.) is a new formulation of doxorubicin. Pegylation protects the liposomes from detection by the mononuclear phagocyte system and increases circulation time, allowing for more targeted delivery of doxorubicin to the tumor cells.

Pegylated liposomal doxorubicin has demonstrated efficacy as a single agent in patients with metastatic or recurrent breast cancer, with objective response rates ranging from 9% to 33% (4, 5). In comparison with conventional doxorubicin, pegylated liposomal doxorubicin has a similar efficacy profile and an improved safety profile, with a significantly reduced incidence of cardiotoxicity and significantly fewer cardiac events, as well as a reduced incidence of myelosuppression, mucositis, nausea, vomiting, and alopecia.

On the other hand, pegylated liposomal doxorubicin is associated with palmar plantar erythema (PPE=hand-foot syndrome), a toxicity rarely or never seen with free doxorubicin.

In addition to its use in breast cancer, liposomal doxorubicin plays a well established role in the treatment of Kaposi's sarcoma (6,7) and recurrent ovarian cancer (8), and has also been successfully used in patients with different types of lymphomas, multiple myeloma, soft tissue sarcoma, glioma, melanoma, mesothelioma, transitional cell carcinoma of the urothelial tract, and in endometrial, pancreatic, gastric, small-cell and non-small-cell lung, hepatocellular, endometrial, renal cell, head and neck, and cholangiocarcinoma (overview in: (9)).

For preclinical studies anti-EGFR immunoliposomes were constructed by using Fab′ fragments of the chimeric MAb cetuximab (C225, cetuximab, erbitux, ImClone Systems Corp., NY, USA; Merck KGaA, Darmstadt, Germany), which were covalently conjugated to the liposome membrane. This approach was designed to provide maximal drug delivery to cancer cells via a receptor-targeted and internalizing drug carrier that is stable, non-immunogenic, long-lived with extended blood and tissue residence times and capable of delivering large payloads of diverse types of drugs. In parallel with MAb fragment optimization, conjugation methodology was also optimized. A new micellar incorporation method was developed involving 2-step conjugation of MAb fragments to preformed drug loaded liposomes (10). First, MAb fragments (Fab′) were covalently conjugated to derivatized PEG-phosphatidyl-ethanolamine (MAL-PEG-DSPE) linkers in solution, resulting in immunoconjugates prone to spontaneous micelle formation. Next, the conjugates were incorporated into drug-preloaded liposomes by controlled heating, resulting in MAb fragments covalently conjugated to the termini of PEG chains and anchored to the liposome.

When Fab′ of C225 was present at only moderate density on immunoliposomes (30 Fab′ per liposome), these immunoliposomes displayed highly efficient binding and internalization in a panel of EGFR or EGFRvIII overexpressing cancer cell lines, as indicated by fluorescence microscopy and FACS (11). These included epidermoid cancer cells (A431), breast cancer cells (MDA-MB-468), malignant glioma cells (U87), and EGFRvIII stable transfectants NR6-M cells. In contrast, irrelevant immunoliposomes (anti-HER2) and control liposomes (no MAb) did not bind to or accumulate in A431, MDA-MB468, U87 or NR6-M cells. Also, anti-EGFR immunoliposomes did not detectably bind to or accumulate in non-EGFR-overexpressing cells (breast cancer cell lines SKBR-3 or MCF-7).

Under in vitro conditions, quantitative studies of immunoliposome uptake, internalization, and intracellular drug delivery were performed using anti-EGFR immunoliposomes loaded with the pH-sensitive probe (HPTS). This method allows quantitative analysis of the kinetics of immunoliposorne uptake at neutral pH (surface-bound) versus at acidic pH (endocytosis-associated) (12). In MDA-MB-468 cells, anti-EGFR immunoliposomes bound within 5 minutes, followed by intracellular accumulation beginning at 15 minutes and increasing up to 240 minutes. Total uptake of EGFR targeted immunoliposomes in MDA-MB-468 cells when present at saturating concentrations was 1.70 fmol phospholipid/cell, which corresponds to uptake of 13,000 liposomes/cell. Uptake of non-targeted liposomes in MDA-MB-468 cells was <300 liposomes/cell, indicating a >43-fold increase due to targeted delivery. Uptake of anti-EGFR immunoliposomes in non-EGFR overexpressing MCF-7 cells was 450 ILs/cell, indicating a 28-fold greater accumulation in EGFR-overexpressing MDA-MB468 cells.

In vivo, anti-EGFR immunoliposomes (ILs) showed extremely long circulation as stable constructs in normal adult rats following a single i.v. dose, with pharmacokinetics that were indistinguishable from those of sterically stabilized (“stealth”) liposomes (13). Moreover, repeat administrations revealed no increase in clearance, further confirming that immunoliposomes retain the long circulation and non-immunogenicity characteristic of stealth liposomes. The potential therapeutic efficacy of anti-EGFR immunoliposomes loaded with a variety of anti-cancer agents (C225-ILs-dox) was evaluated in a series of tumor xenograft models (MDA-MB-468, U-87 and U-87vIII) (13).

The feasibility of the anti-EGFR immunoliposornal system (ILs) for use in human therapy in a clinical set-up has not been demonstrated yet. One of the main concerns in this regard relates to the known toxicities of anti-EGFR immunoliposomes such as, for example, liposomally encapsulated doxorubicin (Doxil, Caelyx). Here the most prominent toxic side-effect is palmar plantar erythema (PPE=hand foot syndrome), which can be observed at a dosage of 40-50 mg/m2 in form of a short infusion every 4 weeks, which is standard in routine oncology practice. Similarly, an important side effect of anti-EGFR antibodies such as Cetuximab is skin toxicity, usually manifesting itself as an acneiform rash of the face and trunk. This side effect is probably a consequence of the fact that the epidermis expresses EGFR at a relatively high level. Therefore, one of the main safety concerns of using anti-EGFR immunoliposomes in a clinical set-set up is that directing said liposomes to EGFR-overexpressing cells via an anti-EGFR antibody such as, for example, Cetuximab might also increase the skin toxicity of the drug.

Further, it has also not yet been demonstrated and is very much unpredictable, whether an anti-EGFR immunoliposome (ILS) encapsulating a chemotherapy drug such as, for example, doxorubicin, vinorelbine or methotrexate, can be used for therapeutic application in a group of patients, which had already received, but not responded to one or multiple standard treatments (first line, second line, third line, etc), in a group of non-responders.

One of the potential reasons for the observed lack of responsiveness in multi-line treatment is the development of a multi-drug resistance of the cancer cells.

Drug resistance continues to be a major challenge in cancer treatment. Intrinsic or acquired drug resistance occurs frequently in most cancers, and often involves resistance to multiple agents simultaneously (multidrug resistance, MDR). A number of mechanisms for drug resistance have been described. These include: overexpressed drug export pumps, such as P-giycoprotein (PGP) and multidrug-resistance protein (MRP); decreased drug uptake, such as altered folate carriers; inactivation of drugs, such as via glutathione-mediated reduction; overexpression of target enzymes, such as upregulated thymidylate synthase; altered drug targets, such as topoisomerase II; increased DNA repair capacity; reduced ability to undergo apoptosis; and others (reviewed in (30) and (31)). Among these mechanisms, the role of PGP in multidrug resistance has been one of the most intensively studied. PGP, encoded by the MDR1 gene, is a member of the ABC (ATP-Binding Cassette) transport protein family and is frequently over-expressed in the MDR phenotype. Other membrane-bound transporters capable of mediating drug efflux include multi-drug resistance protein MRP and other related proteins ((32), (33) and (34)). These proteins actively transport a variety of heterocyclic substrates, including cytotoxic drugs such as anthracyclines, vinca alkaloids, mitoxantrone, paclitaxel, and others out of the cell or into other cellular compartments ((32), (33) and (34)).

Specific inhibitors of these resistance mechanisms have been widely pursued as a means to restore drug sensitivity (for review, see (35)). Although still actively under investigation, specific resistance inhibitors have yet to gain registration for clinical use. Progress towards therapeutic success has been hampered by such issues as inadequate specificity, both predictable and unforeseen toxicities, uncertainty about the true prevalence and contribution of the known resistance mechanisms, paucity of predictive assays to identify tumors dependent upon particular mechanisms, and multiplicity and redundancy of resistance mechanisms ((35)).

There is therefore a need for providing an alternative strategy for a safe therapeutic treatment of patients which have developed cancer, particularly of patients belonging to the group of non-responders, that is patients which are not, or no longer, responsive to a conventional cancer chemotherapy. In particular, there is a need for providing alternative strategies for overcoming intrinsic or acquired drug resistance in cancer therapy.

This need for providing alternative strategies could be satisfied within the scope of the present invention by providing a therapeutic approach which is based on a drug delivery system comprising EGFR-targeted immunoliposomes, which show extensive internalization in the cytoplasm of EGFR-overexpressing cells (up to 30,000 ILs/cell) but not in non-overexpressing cells and also marked cytotoxicity when encapsulating any of several chemotherapy drugs (doxorubicin, vinorelbine and methotrexate).

It was now surprisingly found within the scope of the present invention that administration in a clinical set-up of an anti-EGFR immunoliposome, particularly an immunoliposome comprising any of several chemotherapy drugs such as, for example, doxorubicin, vinorelbine, or methotrexate, to a human patient who is suffering from cancer, particularly a cancer represented by a locally advanced or metastatic tumor, particularly a EGFR-positive tumor, and who is chemotherapy naïve, particularly to a human patient who has received, but not responded or stopped to respond to at least one standard treatment (fist line), particularly to at least two standard treatments (second line), particularly to at least three standard treatments (third line), but especially to all available standard treatments (multi-line), not only resulted in a stabilization of the disease, particularly in a partial response, but especially in a complete response, but also showed no or substantially no side effects, particularly no or substantially no palmar plantar erythema (PPE=hand foot syndrome) and/or skin toxicity.

In one embodiment, an immunoliposome according to the invention and as described herein before is provided comprising an antibody or an antibody fragment, which recognizes and binds to an EGF receptor antigen on the surface of a solid tumor, for first- to multi-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for second-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for third-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for fourth-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for fifth-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for sixth-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for seventh- and higher-line treatment of cancer, particularly a cancer represented by a locally advanced or metastatic tumor, particularly an EGFR-positive tumor, in a human patient in a clinical set-up.

In one embodiment, the invention relates to an immunoliposome according to the invention and as described herein before comprising an antibody or an antibody fragment, which recognizes and binds to an EGF receptor antigen on the surface of a solid tumor, for treatment, particularly for multi-line treatment, of a human patient who has cancer, particularly a cancer represented by a locally advanced or metastatic tumor, particularly a EGFR-positive tumor, and is chemotherapy naïve, particularly a patient, who has received, but not responded to, at least one standard treatment, particularly to at least two standard treatments, particularly to at least three standard treatments, but especially to all available standard treatments.

In one embodiment, the invention relates to an immunoliposome according to the invention and as described herein before for treatment, particularly for multi-line treatment, of a human patient who has a locally advanced or metastatic tumor as described herein before, wherein said tumor is still progressing.

In one embodiment, an immunoliposome according to the invention and as described herein before is provided for treatment, particularly for multi-line treatment, of a human patient who has cancer, particularly a cancer represented by a locally advanced or metastatic tumor as described herein before, wherein the liposome encapsulates an anti-cancer compound, particularly a cytostatic compound, particularly a compound selected from the group consisting of daunomycin, idarubicin, mitoxantrone, mitomycin, cisplatin and other Platinum analogs, vincristine, epirubicin, aclacinornycin, methotrexate, etoposide, doxorubicin, epirubicin, vinorelbine cytosine arabinoside, fluorouracil and other fluorinated pyrimidines, purines, or nucleosides, especially gemcitabine, bleomycin, mitomycin, plicamycin, dactinomycin, cyclophosphamide and derivatives thereof, thiotepa, BCNU, paclitaxel, docetaxel and other taxane derivatives and isolates, camptothecins, polypeptides, a nucleic acid, a nucleic acid having a phosphorothioate internucleotide linkage, and a nucleic acid having a polyamide internucleotide linkage, but especially a compound selected from the group consisting of doxorubicin, epirubicin and vinorelbine, particularly doxorubicin.

in one embodiment, the invention relates to an immunoliposome according to the invention and as described herein before for treatment, particularly for multi-line treatment, of a human patient who has cancer, particularly a cancer represented by a locally advanced or metastatic tumor as described herein before, wherein the non-responsiveness of the patient is caused by multi-drug resistance mechanisms.

In one embodiment, the invention relates to an immunoliposome according to the invention and as described herein before for treatment, particularly for multi-line treatment, of a human patient who has cancer, particularly a cancer represented by a locally advanced or metastatic tumor as described herein before and who has developed a multi drug resistance.

In one embodiment, an immunoliposome according to the invention and as described herein before is provided for treatment, particularly for multi-line treatment, of a human patient belonging to the group of non-responders, who has cancer, particularly a cancer represented by a locally advanced or metastatic tumor as described herein before, particularly to a patient who has developed a multi drug resistance, wherein said immunoliposome has an 1050, determined in a standard MIT assay, of between 1.0 μg/ml and 5.0 μg/ml, particularly of between 0.8 μg/ml and 3.5 μg/ml, particularly of between 0.7 μg/ml and 2.5 μg/ml, particularly of between 0.6 μg/ml and 2.0 μg/ml, particularly of between 0.5 μg/ml and 1.5 μg/ml, particularly of between 0.4 μg/ml and 1.0 μg/ml, particularly of between 0.3 μg/ml and 0.5 μg/ml, particularly of between 0.2 μg/ml and 0.4 μg/ml. The immunoliposome is particularly an immunoliposome comprising doxorubicin.

In one embodiment of the invention, an immunoliposome according to the invention and as described herein before is provided for treatment, particularly for multi-line treatment, of a human patient belonging to the group of non-responders who has cancer, particularly a cancer represented by a locally advanced or metastatic tumor as described herein before, particularly to a patient who has developed a multi drug resistance mechanisms, wherein said immunoliposome has a cytotoxicity which is between 3-fold to 5-fold, between 5-fold to 20-fold, between 10-fold to 30-fold, between 15-fold to 40-fold, between 20-fold to 50-fold, between 25-fold to 60-fold, between 30-fold to 70-fold, between 35-fold to 80-fold, between 40-fold to 90-fold, between 50-fold to 100-fold higher, between 80-fold to 150-fold, between 120-fold to 250-fold higher than that of the free anti-cancer drug.

In one embodiment of the invention, an immunoliposome is provided for treatment, particularly for multi-line treatment, of a human patient who has cancer, particularly a cancer represented by a locally advanced or metastatic tumor as described herein before, particularly a EGFR-positive tumor, wherein said treatment leads to a stabilization of the disease, particularly to a partial response, but especially to a complete response.

In one embodiment of the invention the anti-EGFR immunoliposome is given at a dose level of 10 mg/m² and 40 mg/m² body surface, particularly between 30 mg/m² and 50 mg/m², particularly between 40 mg/m2 and 60 mg/m2, particularly between 50 mg/m2 and 70 mg/m², particularly between 60 mg/m² and 80 mg/m², particularly between 70 mg/m² and 90 mg/m², particularly between 75 mg/m² and 100 mg/m², given as a short infusion every 2 to 6 weeks, particularly every 3 to 5 weeks, but especially every 4 weeks. By a short infusion an infusion time of at least 10 min, particularly of at least 20 min, particularly of at least 30 min, particularly of at least 40 min, particularly of at least 60 min, particularly of at least 90 min, particularly of at least 120 min, particularly of at least 240 min is meant.

In one embodiment, the invention relates to an immunoliposome as described herein before for treatment, particularly for multi-line treatment, of a human patient who is suffering from cancer, particularly a cancer represented by a locally advanced or metastatic tumor, particularly a EGFR-positive tumor, wherein the treatment shows no or substantially no toxic side effects, particularly no or substantially no palmar plantar erythema (PPE=hand foot syndrome) and/or skin toxicity.

In one embodiment, the invention relates to an immunoliposome as described herein before, particularly an immunoliposome comprising a doxorubicin compound, for treatment, particularly for multi-line treatment, of a human patient who is suffering from cancer, particularly a cancer represented by a locally advanced or metastatic tumor, particularly a EGFR-positive tumor, wherein the treatment shows no or substantially no toxic side effects, particularly no or substantially no palmar plantar erythema (PPE=hand foot syndrome) and/or skin toxicity at an immunoliposome concentration of between 5 mg/m² and 20 mg/m² of body surface, particularly between 10 mg/m² and 40 mg/m², particularly between 30 mg/m² and 50 mg/m², particularly between 40 mg/m² and 60 mg/m², particularly between 50 mg/m² and 70 mg/m², particularly between 60 mg/m² and 80 mg/m², particularly between 70 mg/m² and 90 mg/m², particularly between 75 mg/m² and 100 mg/m².

In one embodiment of the invention, an immunoliposome is provided for treatment, particularly for multi-line treatment, of a human patient who has cancer, particularly a cancer represented by a locally advanced or metastatic tumor as described herein before, wherein the antibody or antibody fragment is covalently bound to the liposome membrane, particularly covalently conjugated to the terminus of a linker molecule anchored to the liposome. The linker molecule is particularly a hydrophilic polymer, but especially a polyethylene glycol.

In one embodiment of the invention, the immunoliposome according to the invention and as described herein, which is provided for treatment, particularly for multi-line treatment, of a human patient who has cancer, particularly a cancer represented by a locally advanced or metastatic tumor as described herein before, comprises a monoclonal antibody directed to the ligand-binding extracellular domain of the EGF receptor, particularly a chimeric antibody such as, for example, chimeric MAb C225 or a humanized antibody such as, for example, humanized MAb EMD72000.

In one embodiment, an immunoliposome is provided according to the invention and as described herein before, or a pharmaceutical composition comprising said immunoliposome, wherein the cancer to be treated is a breast, ovarian, cervical, endometrial, gastric, bladder cancer, a uterine sarcoma, a multiple myeloma, and soft tissue and bone sarcomas.

In one embodiment, an immunoliposome according to the invention and as described herein before is provided for treatment, particularly for multi-line treatment, of a human patient in a clinical set-up, wherein said patient is suffering from a cancer selected from the group consisting of Kaposi's sarcoma, recurrent ovarian cancer, soft tissue sarcoma, glioma, melanoma, mesothelioma, transitional cell carcinoma of the urothelial tract, endometrial, pancreatic, small-cell and non-small-cell lung, hepatocellular, renal cell, esophageal, colorectal, anal, vaginal, vulvar, prostate, basal cell carcinoma of the skin head and neck, and cholangio carcinoma, which cancer is particularly represented by a locally advanced or metastatic tumor, particularly a EGFR-positive tumor.

In one embodiment, an immunoliposome is provided according to the invention and as described herein before, or a pharmaceutical composition comprising said immunoliposome, for treatment, particularly multi-line treatment, of a human patient in a clinical set-up, wherein said patient is suffering from a cancer selected from the group consisting of prostate, pancreatic, kidney, oesophageal, colon, and rectal cancer, which cancer is particularly represented by locally advanced or metastatic tumor, particularly a EGFR-positive tumor.

In one embodiment, an immunoliposome is provided according to the invention and as described herein before, or a pharmaceutical composition comprising said immunoliposome, for multi-line treatment, particularly second-line, particularly third line, particularly fourth-line treatment of a human patient in a clinical set-up, wherein said patient is suffering from a prostate cancer with a tumor that has progressed on hormonal and/or docetaxel and/or mitoxanthrone treatment.

In one embodiment, an immunoliposome is provided according to the invention and as described herein before, or a pharmaceutical composition comprising said immunoliposome, for multi-line treatment, particularly for second-line, particularly third line, particularly fourth-line treatment of a human patient in a clinical set-up, wherein said patient is suffering from a pancreatic cancer or a gall bladder cancer with a tumor that has progressed on gemcitabine and/or capecitabine and/or oxaliplatin treatment.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a kidney cancer with a tumor that has progressed on interferon and/or capecitabine and/or sunitinib and/or sorafinib treatment.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a urothelial cancer with a tumor that has progressed on cis- or carboplatinum and/or gemcitabine and/or doxorubicin and/or methotrexate and/or vincristin.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a non-small cell lung cancer with a tumor that has progressed on cis- or carboplatinum and/or gemcitabine and/or vinorelbine and/or, pemetrexed and/or docetaxel and/or gefitinib.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a small cell lung cancer with a tumor that has progressed on cis- or carboplatinum and/or etoposid and/or irinotecan and/or doxorubicin and/or vincristin and/or cyclophosphamide and/or topotecan.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a mesothelioma with a tumor that has progressed on cis- or carboplatinum and/or gemcitabine and/or pemetrexed.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from breast cancer with a tumor that has progressed on on cis- or carboplatinum and/or doxorubicin and/or vincristin and/or cyclophosphamide and/or paclitaxel and/or docetaxel and/or gemcitabine and/or vinorelbine and/or capecitabine and/or mitarnycin and/or methotrexate and/or mitoxanthrone and/or bevacizumab and/or trastuzumab.

In one embodiment, an immunoliposome is provided according to the invention and as described herein before, or a pharmaceutical composition comprising said immunoliposome, for multi-line treatment, particularly for second-line, particularly third line, particularly fourth-line treatment of a human patient in a clinical set-up, wherein said patient is suffering from a esophageal cancer with a tumor that has progressed on cisplatinum and/or 5-FU and/or docetaxel and/or cetuximab treatment.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line treatment of a human patient in a clinical set-up, wherein said patient is suffering from a brain tumor that has progressed on temozolomide and/or bevacizumab and/or irinotecan and/or vincristin and/or procarbacine and/or CCNU and/or BCNU.

In one embodiment, an immunoliposome is provided according to the invention and as described herein before, or a pharmaceutical composition comprising said immunoliposome, for multi-line treatment, particularly for second-line, particularly third line treatment of a human patient in a clinical set-up, wherein said patient is suffering from a hepatocellular cancer with a tumor that has progressed on sunitinib and/or sorafenib.

In one embodiment, an immunoliposome is provided according to the invention and as described herein before, or a pharmaceutical composition comprising said immunoliposome, for multi-line treatment, particularly for second-line, particularly third line, particularly fourth-line, particularly fifth-line, particularly sixth-line, particularly seventh-line treatment of a human patient in a clinical set-up, wherein said patient is suffering from a colon and/or rectal cancer with a tumor that has progressed on cetuximab and/or Bevacizumab and/or oxaliplatin and/or irinotecan and/or capecitabine and/or 5-FU treatment.

In one embodiment of the invention, an immunoliposome is provided for treatment, particularly for multi-line treatment, of a human patient who has cancer, particularly a cancer represented by a locally advanced or metastatic tumor as described herein before, wherein a response rate is achieved of between 5% and 10%, particularly of between 7% and 15%, particularly of between 9% and 20%, particularly between 12% and 25%, particularly between 18% and 30%, particularly between 22% and 35%, particularly between 28% and 40%, particularly between 32% and 45%, particularly between 38% and 50%, particularly between 42% and 55%, particularly between 48% and 60%, particularly between 52% and 60%, particularly between 52% and 70%, particularly between 52% and 75%, particularly between 58% and 80%, particularly between 62% and 85%, particularly between 68% and 90%, particularly between 72% and 95%, and up to 100%.

In one embodiment, the invention relates to a pharmaceutical composition comprising an immunoliposome according to the invention and as disclosed herein before, together with a pharmaceutically acceptable carrier or excipient or a diluent, for first- to multi-line, particularly for second-line, particularly third-line, particularly fourth-line, particularly fifth-line, particularly sixth-line, particularly seventh- and higher-line treatment of cancer, particularly a cancer represented by a locally advanced or metastatic tumor, particularly an EGFR-positive tumor, in a human patient in a clinical set-up, particularly a human patient belonging to the group of non-responders, particularly a human patient belonging to the group of non-responders who has developed a multidrug resistance.

In one embodiment, the invention relates to a method of first- to multi-line, particularly of second-line, particularly of third-line, particularly of fourth-line, particularly of fifth-line, particularly of sixth-line, particularly of seventh- and higher-line treatment of cancer, particularly a cancer represented by a locally advanced or metastatic tumor, particularly an EGFR-positive tumor, in a human patient, particularly a human patient belonging to a group of non-responders, particularly in a human patient belonging to the group of non-responders who has developed a multidrug resistance, in a clinical set-up by administering to said human patient an immunoliposorne or a pharmaceutical composition according to the invention and as disclosed herein before.

In one embodiment, the invention relates to a method of treating a human patient who has cancer, particularly a cancer represented by a locally advanced or metastatic tumor, particularly a EGFR-positive tumor, and is chemotherapy naïve, particularly a patient, who has received, but not responded to, at least one standard treatment, particularly to at least two standard treatments, particularly to at least three standard treatments, but especially to all available standard treatments with an immunoliposome or a pharmaceutical composition according to the invention and as disclosed herein before.

In particular, the invention relates to a method of treating a human patient who has developed a multi-drug resistance.

In one embodiment, the invention relates to a method of using an immunoliposome or a pharmaceutical composition according to the invention and as disclosed herein before for the preparation of a medicament for use in first- to multi-line, particularly second-line, particularly third-line, particularly fourth-line, particularly fifth-line, particularly sixth-line, particularly seventh- and higher-line treatment of cancer, in a clinical set-up, particularly a cancer represented by a locally advanced or metastatic tumor, particularly an EGFR positive tumor, in a human patient, particularly in a human patient belonging to the group of non-responders, particularly in a human patient belonging to the group of non-responders who has developed a multidrug resistance.

In one embodiment, the invention relates to a method of using an immunoliposome or a pharmaceutical composition according to the invention and as disclosed herein before for the preparation of a medicament for use in the treatment of a human patient who has cancer, particularly a cancer represented by a locally advanced or metastatic tumor, particularly a EGFR-positive tumor, and is chemotherapy naive, particularly a patient who has received, but not responded to, at least one standard treatment, particularly to at least two standard treatments, particularly to at least three standard treatments, but especially to all available standard treatments.

In particular, the invention relates to a method of using an immunoliposorne or a pharmaceutical composition according to the invention and as disclosed herein before, for the preparation of a medicament for use in the treatment of a human patient who has developed a multi-drug resistance.

In one embodiment, an immunoliposome is provided according to the present invention and as described herein comprising an antibody or an antibody fragment, which recognizes and binds to an EGF receptor antigen on the surface of a solid tumor and further encapsulating in the liposome an anti-tumor compound, or a pharmaceutical composition comprising such an immunoliposome, for the treatment of multi-drug resistance in a patient or a group of patients which have developed such a multi-drug resistance.

In one embodiment, the invention relates to a pharmaceutical composition comprising an immunoliposome according to the present invention and as described herein together with a pharmaceutically acceptable carrier or excipient or a diluent for the treatment of cancer, particularly for the treatment of breast cancer or a colonrectal cancer, or both, in a patient or a group of patients who have developed a multi-drug resistance, particularly a multi-drug resistance against treatment with one or more anti-cancer drugs selected from the group consisting of docetaxel, mitoxanthrone, gemcitabine, capecitabine, oxaliplatin, interferon, sunitinib, sorafinib, cis- or carboplatinum, doxorubicin, methotrexate, vincristin, vinorelbine, pemetrexed, gefitinib, etoposid, irinotecan, cyclophosphamide, topotecan, cyclophosphamide, paclitaxel, mitomycin, bevacizumab, trastuzumab, 5-FU, cetuximab, temozolomide, bevacizumab, procarbacine, CCNU, and BCNU.

In one embodiment, said multi-drug resistance comprises one or more anti-cancer drugs selected from the group consisting of docetaxel, mitoxanthrone, gemcitabine, capecitabine, oxaliplatin, sunitinib, sorafinib, cisplatinum, 5-FU, cetuximab, Bevacizumab, oxaliplatin and irinotecan.

In one embodiment, a pharmaceutical composition is provided comprising an immunoliposome according to the present invention and as described herein together with a pharmaceutically acceptable carrier or excipient or a diluent for treatment, particularly for multi-line treatment, of cancer, particularly for the treatment of breast cancer or a colonrectal cancer, or both, wherein said immunoliposome encapsulates doxorubicin and further comprises antibody MAb 0225 or antibody EMD72000 or a fragment thereof, which still exhibits the specific binding properties of one or both of said antibodies.

In one embodiment, a pharmaceutical composition is provided comprising an immunoliposome according to the present invention and as described herein together with a pharmaceutically acceptable carrier or excipient or a diluent for the treatment of cancer, particularly for the treatment of breast cancer or a colonrectal cancer, or both, in a patient or a group of patients who have developed a multi-drug resistance, particularly a multi-drug resistance against treatment with one or more anti-cancer drugs selected from the group consisting of docetaxel, mitoxanthrone, gemcitabine, capecitabine, oxaliplatin, interferon, sunitinib, sorafinib, cis- or carboplatinum, doxorubicin, methotrexate, vincristin, vinorelbine, pemetrexed, gefitinib, etoposid, irinotecan, cyclophosphamide, topotecan, cyclophosphamide, paclitaxel, mitomycin, bevacizumab, trastuzumab, 5-FU, cetuximab, temozolomide, bevacizumab, procarbacine, CCNU, and BCNU, wherein said immunoliposome encapsulates doxorubicin and further comprises antibody MAb C225 or antibody EMD72000 or a fragment thereof, which still exhibits the specific binding properties of one or both of said antibodies.

### DEFINITIONS

The term “comprise” is generally used in the sense of include, that is to say permitting the presence of one or more features or components.

As used in the specification and claims, the singular form “a”, “an” and “the” include plural references unless the context clearly dictates otherwise. For example, the term “a patient” includes a plurality of patients. The term “an immunoliposome” includes a plurality of immunoliposomes, including mixtures thereof.

The term “EGF Receptor” or “EGFR”, “ErbB1”, “HER1” is an art recognized term and used herein synonymously and is understood to refer to a receptor protein which is a member of the class I family of Receptor Tyrosine Kinases (RTKs), which includes EGFR (ErbB1, HER1), HER2 (ErbB2), HER3 (ErbB3) and HER4 (ErbB4). As a target antigen, EGFR is a readily accessible cell surface receptor, which is overexpressed in many human solid tumors. Also included in this definition are mutants of EGFR, particularly Class III mutants such as, for example, EGFRvIII, which contains a deletion in exons 2-7 within the ECD, resulting in an in-frame deletion of 801 by of the coding sequence and the generation of a novel glycine residue at the fusion junction.

The term “first-line treatment” or “first-line therapy” as used herein is an art recognized term and is understood to refer to the first chemotherapy treatment of cancer, which may be combined with surgery and/or radiation therapy, also called primary treatment or primary therapy.

The term “second-line treatment” or “second-line therapy” as used herein is an art recognized term and is understood to refer to a chemotherapy treatment that is given when initial or primary treatment (first-line or primary therapy) doesn't work, or stops working.

The term “third-line, fourth-line, fifth-line, etc, treatment” or “third-line, fourth-line, fifth-line, etc, therapy” as used herein is an art recognized term and is understood to refer to a chemotherapy treatment that is given when initial treatment and any of the following treatments (first-line, second-line, third-line, etc, therapy) doesn't work, or stops working.

The term “multi-line” treatment is a general term and understood herein to refer to any higher-line treatment that follows an initial or primary treatment (first-line or primary therapy), which doesn't work, or has stopped working.

The term “substantially no side effect” or “substantially no adverse side effect” as used herein is an art recognized term and understood to refer to mild to moderate drug-related effects or toxicities, which are not dose limiting.

The term “EGFR-positive tumor” as used herein is understood to refer to a tumor that contains at least 1%, particularly at least 2%, 3%, 4% or 5%, particularly at least 10%, EGFR positive cells, detected e.g. by an immunohistochemistry test such as, for example, the FDA approved EGFR pharmaDx kit (“DAKO” test; DAKO Notrth America, Inc), the Zymed EGFR kit or the Ventana EGFR 3C6 antibody, In particular, said EGFR positive cells overexpress the EGFR antigen and/or mutants of EGFR, particularly Class III mutants such as, for example, EGFRvIII.

“A pharmaceutically effective amount” refers to a chemical material or compound which, when administered to a human or animal organism, induces a detectable pharmacologic and/or physiologic effect.

The respective pharmaceutically effect amount can depend on the specific patient to be treated, on the disease to be treated and on the method of administration. Further, the pharmaceutically effective amount depends on the specific protein used, especially if the protein additionally contains a drug as described or not. The treatment usually comprises a multiple administration of the pharmaceutical composition, usually in intervals of several hours, days or weeks. The pharmaceutically effective amount of a dosage unit of the immunoliposome according to the present invention usually is in the range of between 5 mg/m² and 100 mg/m² of body surface of the patient to be treated.

The phrase “pharmaceutically acceptable” refers to molecular entities and compositions that are physiologically tolerable and do not typically produce an allergic or similar untoward reaction, such as gastric upset, dizziness and the like, when administered to a human.

The terms “antibody” or “antibodies” as used herein is an art-recognized term and is understood to refer to molecules or active fragments of molecules that bind to known antigens, particularly the terms “antibody” or “antibodies” refer to immunoglobulin molecules and to immunologically active portions of immunogiobulin molecules, i.e molecules that contain a binding site that immunospecifically binds an antigen. The immunoglobulin according to the invention can be of any type (IgG, IgM, IgD, IgE, IgA and IgY) or class (IgG1, IgG2, IgG3, IgG4, IgA1 and IgA2) or subclasses of immunoglobulin molecule.

“Antibodies” are intended within the scope of the present invention to include monoclonal antibodies, polyclonal, chimeric, single chain, bispecific, simianized, human and humanized antibodies as well as active fragments thereof. The term “fragment” refers to a part or portion of an antibody or antibody chain comprising fewer amino acid residues than an intact or complete antibody or antibody chain. Examples of active fragments of molecules that bind to known antigens include separated light and heavy chains, Fab, Fab/c, Fv, Fab′, and F(ab′)₂ fragments, including the products of an Fab immunoglobulin expression library and epitope-binding fragments of any of the antibodies and fragments mentioned above. Fragments can be obtained via chemical or enzymatic treatment of an intact or complete antibody or antibody chain. Fragments can also be obtained by recombinant means. Exemplary fragments include Fab, Fab′, F(ab′)₂, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding).

Antibody-binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies.

These active fragments can be derived from a given antibody by a number of techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example (14); (15).

A “chimeric antibody” is an antibody in which one or more regions of the antibody are from one species of animal and one or more regions of the antibody are from a different species of animal. A preferred chimeric antibody is one which includes regions from a primate immunoglobulin. A chimeric antibody for human clinical use is typically understood to have variable regions from a non-human animal, e.g. a rodent, with the constant regions from a human. In contrast, a humanized antibody uses CDRs from the non-human antibody with most or all of the variable framework regions from and all the constant regions from a human immunoglobulin. A human chimeric antibody is typically understood to have the variable regions from a rodent. A typical human chimeric antibody has human heavy constant regions and human light chain constant regions with the variable regions of both the heavy and light coming from a rodent antibody. A chimeric antibody may include some changes to a native amino acid sequence of the human constant regions and the native rodent variable region sequence. Chimeric and humanized antibodies may be prepared by methods well known in the art including CDR grafting approaches (see, e.g., U.S. Pat. Nos. 5,843,708; 6,180,870; 5,693,762; 5,585,089; 5,530,101), chain shuffling strategies (see e.g., U.S. Pat. No. 5,565,332; (16), molecular modelling strategies (U.S. Pat. No. 5,639,641), and the like.

A “humanized antibody” refers to a type of engineered antibody which incorporates at least one humanized immunoglobulin or antibody chain or fragment thereof, particularly at least one humanized light or heavy chain. Said humanized immunoglobulin or antibody chain or fragment thereof, but particularly the at least one humanized light or heavy chain is derived from a non-human source, particularly a non-human antibody, typically of rodent origin. Said non-human contribution to the humanized antibody is typically provided in form of at least one CDR region which is interspersed among framework regions derived from one (or more) human immunoglobulin(s). In addition, framework support residues may be altered to preserve binding affinity.

The humanized antibody may further comprise constant regions (e.g., at least one constant region or portion thereof, in the case of a light chain, and preferably three constant regions in the case of a heavy chain).

Methods to obtain “humanized antibodies” are well known to those skilled in the art. (17).

A “humanized antibody” may also be obtained by a novel genetic engineering approach that enables production of affinity-matured human-like polyclonal antibodies in large animals such as, for example, rabbits (http://www.rctech.com/bioventures/-therapeutic.php).

The term “immunoliposome dosage” or “immunoliposome concentration” generally refers to the concentration of the anti-cancer agent entrapped in the liposome.

A “liposome” refers to a small, spherical vesicle composed of lipids, particularly vesicle-forming lipids capable of spontaneously arranging into lipid bilayer structures in water with its hydrophobic moiety in contact with the interior, hydrophobic region of the bilayer membrane, and its head group moiety oriented toward the exterior, polar surface of the membrane. Vesicle-forming lipids have typically two hydrocarbon chains, particularly acyl chains, and a head group, either polar or nonpolar. Vesicle-forming lipids are either composed of naturally-occurring lipids or of synthetic origin, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids for use in the composition of the present invention include glycolipids and sterols such as cholesterol and its various analogs which can also be used in the liposomes.

Cationic lipids, which typically have a lipophilic moiety, such as a sterol, an acyl or diacyl chain, and where the lipid has an overall net positive charge can also be suitably used in liposomes. The head group of the lipid typically carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly]cholesterol (DC-Chol); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids.

The liposomes can include a vesicle-forming lipid derivatized with a hydrophilic polymer to form a surface coating of hydrophilic polymer chains on the liposomes surface. A vesicle-forming lipid, in particular a phospholipid, such as distearoyl phosphatidylethanolamine (DSPE), may be covalently attached to a hydrophilic polymer, which forms a surface coating of hydrophilic polymer chains around the liposome. Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

A preferred hydrophilic polymer chain is polyethyleneglycol (PEG), preferably as a PEG chain having a molecular weight between 200-20,000 daltons, more preferably between 500-10,000 daltons, still more preferably between 750-5000 daltons. Methoxy or ethoxy-capped analogues of PEG are also preferred hydrophilic polymers, commercially available in a variety of polymer sizes, e.g., 120-20,000 Daltons.

Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of liposome formation is typically achieved by including between 0.5-20 mole percent of the polymer-derivatized lipid with the remaining liposome forming components, e.g., vesicle-forming lipids.

Preparation of Vesicle-Forming Lipids Derivatized with Hydrophilic Polymers has been described, for example in U.S. Pat. No. 5,395,619, in U.S. Pat. No. 5,013,556, in U.S. Pat. No. 5,631,018 and in WO 98/07409. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

An “internalizing antibody” is an antibody that, upon binding to a receptor or other ligand on a cell surface, is transported into the cell, for example, into a lysozyme or other organelle or into the cytoplasm.

The present invention relates to an immunoliposome comprising an antibody or an antibody fragment, which recognizes and binds to an EGF receptor antigen on the surface of a solid tumor and comprises at least one anti-tumor agent, for first- to multi-line, particularly to second-line, particularly to third line, particularly to fourth-line, particularly to fifth-line, particularly to sixth-line, particularly to seventh- and higher-line treatment of cancer, particularly a cancer represented by a locally advanced or metastatic tumor, particularly an EGFR-positive tumor, in a human patient in a clinical set-up.

The immunoliposome composition of the invention thus also includes an antibody or antibody fragment including Fab, Fab′, F(ab′)₂, Fabc, Fv, single chains, and single-chain antibodies that specifically recognizes and bind to EGF receptor on the surface of a tumor derived cell. In another embodiment, the antibody comprises at least one binding domain which specifically binds the EGR receptor on the surface of a tumor-derived cell. In an alternate embodiment, the antibody is a single chain antibody comprising at least one binding domain which specifically binds EGF receptor on the surface of a tumor-derived cell.

Antibodies may be attached to a liposome by covalent methods known in the art. For attaching an antibody covalently to a liposome, a derivatized lipid containing an end-functionalized polyethylene glycol chain is incorporated into Liposomes. After liposome formation, the end-functionalized group can react with an antibody for antibody coupling to a liposome.

There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (18; 19; 20; 21; 22).

Generally, the PEG chains are functionalized to contain reactive groups suitable for coupling with, for example, sulfhydryls, amino groups, and aldehydes or ketones (typically derived from mild oxidation of carbohydrate portions of an antibody) present in a wide variety of ligands. Examples of such PEG-terminal reactive groups include maleimide (for reaction with sulfhydryl groups), N-hydroxysuccinimide (NHS) or NHS-carbonate ester (for reaction with primary amines), hydrazide or hydrazine (for reaction with aldehydes or ketones), iodoacetyl (preferentially reactive with sulfhydryl groups) and dithiopyridine (thiol-reactive). Liposomes carrying an entrapped agent and bearing surface-bound targeting ligands, i.e., targeted, therapeutic liposomes, are prepared by any of these approaches. A preferred method of preparation is the insertion method, where preformed liposomes and are incubated with the targeting conjugate to achieve insertion of the targeting conjugate into the liposomal bilayers. In this approach, liposomes are prepared by a variety of techniques, such as those detailed in (23), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns. The liposomes can include a vesicle-forming lipid derivatized with a hydrophilic polymer to form a surface coating of hydrophilic polymer chains on the liposomes surface. Addition of a lipid-polymer conjugate is optional, since after the insertion step, described below, the liposomes will include lipid-polymer-targeting ligand. Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of Liposome formation is typically achieved by including between 0.5-20 mole percent of the polymer-derivatized lipid with the remaining liposome forming components, e.g., vesicle-forming lipids. Exemplary methods of preparing polymer-derivatized lipids and of forming polymer-coated liposomes have been described in U.S. Pat. Nos. 5,013,556, 5,631,018 and 5,395,619, which are incorporated herein by reference. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

Alternatively, an antibody-lipid derivative may be first formed and then incorporated into a liposome. As an example, an antibody is coupled to the maleimide group of a free DSPE-PEG molecule. The antibody-coupled DSPE-PEG molecule is then employed to form vesicles.

After formation of the liposomes, a targeting ligand is incorporated to achieve a cell-targeted, therapeutic liposome. The targeting ligand is incorporated by incubating the pre-formed liposomes with the lipid-polymer-ligand conjugate, prepared as described above. The pre-formed liposomes and the conjugate are incubated under conditions effective to association with the conjugate and the liposomes, which may include interaction of the conjugate with the outer liposome bilayer or insertion of the conjugate into the liposome bilayer. More specifically, the two components are incubated together under conditions which achieve associate of the conjugate with the liposomes in such a way that the targeting ligand is oriented outwardly from the liposome surface, and therefore available for interaction with its cognate receptor. It will be appreciated that the conditions effective to achieve such association or insertion are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

The targeting conjugate, in the form of a lipid-polymer-ligand conjugate, will typically form a solution of micelles when the conjugate is mixed with an aqueous solvent. The micellar solution of the conjugates is mixed with a suspension of pre-formed liposomes for incubation and association of the conjugate with the liposomes or insertion of the conjugate into the liposomal lipid bilayers. The incubation is effective to achieve associate or insertion of the lipid-polymer-antibody conjugate with the outer bilayer leaflet of the liposomes, to form an immunoliposome.

After preparation, the immunoliposomes preferably have a size of less than about 200 nm, preferably of between about 85-120 nm, and more preferably of between 90-110 nm, as measured, for example, by dynamic light scattering at 30[deg.] or 90[deg.].

Liposome compositions are typically prepared with lipid components present in a molar ratio of about 30-75 percent vesicle-forming lipids, 25-40 percent cholesterol, 0.5-20 percent polymer derivatized lipid, and 0.0001-10 mole percent of the lipid derivative employed for antibody coupling.

Generally, a therapeutic drug is incorporated into liposomes by adding the drug to the vesicle forming lipids prior to liposome formation, as described below, to entrap the drug in the formed liposome. If the drug is hydrophobic the drug is added directly to the hydrophobic mixture. If the drug is hydrophilic the drug can be added to the aqueous medium which covers the thin film of evaporated lipids.

The liposomes to be used in the present invention include an anti-tumor agent. Antitumor compounds contemplated for use in the invention include, but are not limited to, plant alkaloids, such as vincristine, vinblastine and etoposide; anthracycline antibiotics including doxorubicin, epirubicin, daunorubicin; fluorouracil; antibiotics including bleomycin, mitomycin, plicamycin, dactinomycin; topoisomerase inhibitors, such as camptothecin and its analogues; and platinum compounds, including cisplatin and its analogues, such as carboplatin. Other traditional chemotherapeutic agents suitable for use are known to those of skill in the art and include, asparaginase, busulfan, chlorambucil, cyclophosphamide, cytarabine, dacarbazine, estramustine phosphate sodium, floxuridine, fluorouracil (5-FU), hydroxyurea (hydroxycarbamide), ifosfamide, lomustine (CCNU), mechlorethamine HCl (nitrogen mustard), melphalan, mercaptopurine, methotrexate (MTX), mitomycin, mitotane, mitoxantrone, procarbazine, streptozocin, thioguanine, thiotepa, amsacrine (m-AMSA), azacitidine, hexamethylmelamine (HMM), mitoguazone (methyl-GAG; methyl glyoxal bis-guanylhydrazone; MGBG), semustine (methyl-CCNU), teniposide (VM-26) and vindesine sulfate.

In one embodiment of the invention, the liposomes have a size suitable for extravasation into a solid tumor. This is particularly useful where the liposomes also include a surface coating of a hydrophilic polymer chain to extend the blood circulation lifetime of the liposomes. Liposomes remaining in circulation for longer periods of time, e.g., more than about 2-5 hours, are capable of extravasating into tumors and sites of infection, which exhibit compromised leaky vasculature or endothelial barriers. Such liposomes are typically between about 40-200 nm, more preferably between 50-150 nm, most preferably between 70-120 nm.

The selected agent is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse-phase evaporation, are also suitable.

Alternatively, the drug may be incorporated into preformed liposomes by active transport mechanisms. Typically, in this case drug is taken up in liposomes in response to a potassium or hydrogen ion concentration differential (Mayer, 1986; Mayer 1989).

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (24).

Liposomes carrying an entrapped agent and bearing surface-bound targeting ligands, i.e., targeted, therapeutic liposomes, may be prepared by any of these approaches. A preferred method of preparation is the insertion method, where pre-formed liposomes and are incubated with the targeting conjugate to achieve insertion of the targeting conjugate into the liposomal bilayers. In this approach, liposomes are prepared by a variety of techniques, such as those detailed in (23), and specific examples of liposomes prepared in support of the present invention will be described below. Typically, the liposomes are multilamellar vesicles (MLVs) or unilamellar vesicles (ULVs).

MLVs can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

ULVs can be formed by the repeated freeze-thawing method. In this method 1-2-oleoyl-3-sn-glycerophosphocholine and Chol, or DSPC and Choi (molar ratio 3:2) is mixed with mPEGDSPE (0.6-5 mol % of phospholipid). Liposomes are subsequently extruded several times through polycarbonate filters with defined pore sizes of 0.1, 0.08 and 0.05 μm. This yields liposomes typically with sizes of 70-120 nm diameters. The size of the liposomes may be determined by dynamic light scattering. Liposome concentration can be measured using a standard phosphate assay.

The anti-EGFR immunoliposomes obtainable by any of the above described methods has clinical relevance and can be used in second and higher-fine treatment of human patients suffering from cancer, particularly a cancer represented by a locally advanced or metastatic tumor. The immunoliposome contemplated for use in the present invention comprises an antibody or an antibody fragment, which recognizes and binds to an EGF receptor antigen on the surface of a solid tumor. In particular, the immunoliposome comprises a Fab, Fab′, F(ab′)₂, Fabc, Fv fragment, or is a single-chain antibody.

The immunoliposome contemplated for use in the present invention further comprises an anti-tumor agent, particularly anti-tumor agent selected from the group consisting of doxorubicin, epirubicin and vinorelbine, particularly doxorubicin.

The immunoliposome according to the invention may be administered to a human patient in form of a pharmaceutical composition comprising said immunoliposome together with a pharmaceutically acceptable carrier and/or a diluent and/or an excipient. Formulation of the pharmaceutical composition according to the invention can be accomplished according to standard methodology known to those skilled in the art.

The immunoliposome according to the invention or a pharmaceutical compositions comprising said immunoliposome may be administered to a subject in the form of a solid, liquid or aerosol at a suitable, pharmaceutically effective dose. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for infusions, formulations adapted for injection intramuscularly, subcutaneously, intravenously, intra-arterially, and formulations for topical and intraocular administration. Examples of aerosol formulations include inhaler formulations for administration to the lungs.

The immunoliposome according to the invention or a pharmaceutical compositions comprising said immunoliposome may be administered by standard routes of administration. In general, the composition may be administered by topical, oral, rectal, nasal, interdermal, intraperitoneal, or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

It is well known to those skilled in the pertinent art that the dosage of a pharmaceutical composition will depend on various factors such as, for example, the condition of being treated, the particular composition used, and other clinical factors such as weight, size, sex and general health condition of the patient, body surface area, the particular compound or composition to be administered, other drugs being administered concurrently, and the route of administration.

The immunoliposome according to the invention or the composition comprising said immunoliposome may be administered in combination with an biologically active substance or compound or other compositions comprising said biologically active substance or compound, particularly an anti-tumor compound, particularly at least one cytostatic compound, particularly a compound selected from the group consisting of particularly a compound selected from the group consisting of daunomycin, idarubicin, mitoxantrone, mitomycin, cisplatin and other Platinum analogs, vincristine, epirubicin, aclacinornycin, methotrexate, etoposide, doxorubicin, cytosine arabinoside, fluorouracil and other fluorinated pyrimidines, purines, or nucleosides, especially gemcitabine, bleomycin, mitomycin, plicamycin, dactinomycin, cyclophosphamide and derivatives thereof, thiotepa, BCNU, paclitaxel docetaxel and other taxane derivatives and isolates, camptothecins, polypeptides, a nucleic acid, a nucleic acid having a phosphorothioate internucleotide linkage, and a nucleic acid having a polyamide internucleotide linkage, but especially doxorubicin, epirubicin and vinorelbine, together with an antibody according to the present invention and, optionally, a pharmaceutically acceptable carrier and/or a diluent and/or an excipient.

Pharmaceutically active matter, particularly the anti-tumor compounds which are entrapped in the immunoliposome, may be present in amounts between 0.1 mg/m² ng and 2.5 g/m² of body surface and per dose. Generally, the regime of administration should be in the range of between 0.5 mg/m² and 1000 mg/m² of the anti-tumor compound according to the invention, particularly in a range of between 1.0 mg/m² to 500 mg/m², and particularly in a range of between 5.0 mg/m² and 250 mg/m², particularly in a range of between 10.0 mg/m² and 150 mg/m², with all individual numbers falling within these ranges also being part of the invention, lithe administration occurs through continuous infusion a more proper dosage may be in the range of between 0.01 μg and 10 mg units per kilogram of body weight per hour with all individual numbers falling within these ranges also being part of the invention.

The antibody concentration of the immunoliposome is in a range of between 1 μg to 150 μg of antibody or antibody fragment per μmol phospholipid, particularly in a range of 5 μg to 100 μg of antibody or antibody fragment per μmol phospholipid, particularly in a range of 10 μg to 100 μg of antibody or antibody fragment per μmol phospholipid, particularly in a range of 20 μg to 50 μg of antibody or antibody fragment per μmol phospholipid, particularly in a range of 30 μg to 40 μg of antibody or antibody fragment per μmol phospholipid.

The immunoliposomal preparation of the present invention may be prepared in the form of a pharmaceutical composition containing the isolated and purified immunoliposome dissolved or dispersed in a pharmaceutically acceptable carrier well known to those skilled in the art, for parenteral administration by, e.g., intravenous, subcutaneous or intramuscular injection or by intravenous drip infusion.

As to a pharmaceutical composition for parenteral administration, any conventional additives may be used such as excipients, adjuvants, binders, disintegrants, dispersing agents, lubricants, diluents, absorption enhancers, buffering agents, surfactants, solubilizing agents, preservatives, emulsifiers, isotonizers, stabilizers, solubilizers for injection, pH adjusting agents, etc.

Acceptable carriers, diluents and adjuvants which facilitates processing of the active compounds into preparation which can be used pharmaceutically are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the pharmaceutical composition may be systemic or topical. For example, administration of such a composition may be various parenteral routes such as subcutaneous, intravenous, intradermal, intramuscular, intraperitoneal, intranasal, transdermal, buccal routes or via an implanted device, and may also be delivered by peristaltic means.

Administration will generally be parenterally, eg intravenously, particularly in form of an infusion. Preparations for parenteral administration include sterile aqueous or non aqueous solutions, suspensions and emulsions. Non-aqueous solvents include without being limited to it, propylene glycol, polyethylene glycol, vegetable oil such as olive oil, and injectable organic esters such as ethyl oleate. Aqueous solvents may be chosen from the group consisting of water, alcohol/aqueous solutions, emulsions or suspensions including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose) and others. Preservatives may also be present such as, for example, antimicrobials, anti-oxidants, chelating agents, inert gases, etc.

The pharmaceutical composition may further comprise proteinaceous carriers such as for example, serum albumine or immunoglobuline, particularly of human origin. Further biologically active agents may be present in the pharmaceutical composition of the invention dependent on its intended use.

In one aspect of the invention, immunoliposomes (ILs) as described herein were generated that bind EGFR to provide efficient antibody-directed intracellular delivery of anticancer drugs into target cells to study whether it is possible by this approach to overcome drug resistance mechanisms, which remain an important obstacle towards better outcomes in cancer therapy.

ILs may be constructed modularly with various MAb or MAb fragments, including chimeric antibodies such as, for example, Fab′ from 0225 (cetuximab, Erbitux®) or humanized antibodies, such as, for example, EMD72000, covalently linked to stabilized liposomes containing various drugs or probes.

EGFR-overexpressing cells that also feature mdr-mediated multidrug-resistance such as, for example, human breast cancer cell line MDA-MB-231/mdr or colorectal cancer cell line HT-29/mdr, can then be treated with the so-produced ILs.

In the multidrug resistant cell lines, ILs loaded with doxorubicin (dox) could be shown to produce-15-86-fold greater cytotoxicity than free doxorubicin (e.g. IC50 of ILs-dox in HT-29/mdr cells=0.37 vs. IC50 of free dox=6.0 (μg dox/ml). In non-resistant HT-29 cells immunoliposomal cytotoxicity of doxorubicin was comparable to that of the free drug (IC50=0.23 vs. 0.36 μg dox/ml), while markedly more cytotoxic than the non-targeted liposomal doxorubicin (IC50>27 μg dox/ml). Interestingly, intracellular distribution studies in MDA-MB-231/mdr cells revealed distinctive differences between free dox and immunoliposomal dox delivery. While free dox was efficiently pumped out of this multidrug resistant tumor cells, immunoliposomal dox at the identical concentration reached 3.5-8 times higher accumulation of dox in the cytoplasma and 3.5-4.9 times in the nuclei.

Finally, in vivo therapy studies in the MDA-MB-231/mdr xenograft model confirmed the ability of anti-EGFR ILs-dox to efficiently target multidrug resistant cells. While free dox failed to show any activity at its MTD in this highly multidrug-resistant tumor model, anti-EGFR EGFR ILs-dox showed impressive antitumor effects, clearly superior to all other treatments.

The immunoliposomes according to the present invention and as disclosed herein thus provide efficient and targeted drug delivery to EGFR-overexpressing tumor cells and show potent activity even against multidrug-resistant cells.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

The target population are patients with EGFR-overexpressing solid tumors who have received all available standard treatments.

In particular, the patients are suffering from the following cancers and the tumor has progressed on the following treatments:

Prostate cancer with tumors progressed on hormonal treatment, docetaxel, mitoxanthrone.
Pancreatic and gall bladder cancer with tumors progressed on Gerncitabine, Capecitabine, oxaliplatin
Kidney cancer with tumors progressed on interferon, capecitabine, sunitinib, sorafinib.
Urothelial cancer with tumors progressed on cis- or carboplatinum, gemcitabine, doxorubicin, methotrexate, vincristin.
Non-small cell lung cancer with tumors progressed on cis- or carboplatinum, gemcitabine, vinorelbine, pemetrexed, docetaxel, gefitinib.
Small cell lung cancer with tumors progressed on cis- or carboplatinum, etoposid, irinotecan, doxorubicin, vincristin, cyclophosphamide, topotecan.
Mesothelioma with tumors progressed on cis- or carboplatinum, gemcitabine, pemetrexed.
Breast cancer with tumors progressed on cis- or carboplatinum, doxorubicin, vincristin, cyclophosphamide, paclitaxel, docetaxel, gemcitabine, vinorelbine, capecitabine, mitomycin, methotrexate, mitoxanthrone, bevacizumab, trastuzumab.
Esophageal cancer with tumors progressed on cis- or carboplatinum, 5-FU, docetaxel Head&Neck cancer with tumors progressed on cis- or carboplatinum, 5-FU, docetaxel, cetuximab,
Brain tumors with tumors progressed on temozolomide, bevacizumab, irinotecan, vincristin, procarbacine, CCNU, BCNU.
Hepatocellular cancer with tumors progressed on sunitinib, sorafenib.
Colon and rectal cancer with tumors progressed on Cetuximab, Bevacizumab, oxaliplatin, irinotecan, capecitabine, 5-FU

The therapeutic compound tested in the trial is C225-ILs-dox, a construct in which the EGFR-specific antibody C225 is covalently bound to the lipid membrane of doxorubicin-containing liposomes. The rationale to use this compound is the fact that doxorubicin is one of the most active agents in many human tumors, and that a high percentage of these malignancies do express EGFR.

### A: Protocol of Phase I Study CC1

### 1. Selection Criteria

### 1.1. Total Number of Patients

Approximately 30 patients.

### 1.2. Inclusion Criteria

Prior to enrollment in the study candidates must meet ALL the following criteria:
1. Histologically proven locally advanced or metastatic solid tumor.
2. ECOG Performance ≦2.
3. No additional standard therapy available for the patient.
4. EGFR overexpression (according to DAKO EGFR pharmDx—Test) determined in the most recently evaluable tumor tissue.
5. No concomitant anti-tumor therapy (steroids are permitted—in breast cancer and prostate cancer, steroid dose needs to remain stable during the study period).
6. At least four weeks since termination of any previous anti-tumor treatment (6 weeks in the case of nitrosoureas or mitomycin C).
7. In patients with previous anthracycline exposure, a normal echocardiogram (LVEF >50%) is required.
8. Age ≧18.
9. Male or female.
10. Female and male patients of reproductive age must be using effective contraception.
11. Willing and able to sign an informed consent prior to participation in the study and to comply with, the protocol for the duration of the study.

### 1.3. Exclusion Criteria

Candidates must be excluded from the study if ANY of the following criteria are met:
1. Pregnancy and/or breastfeeding.
2. Patients with the following laboratory values
   neutrophils <1.5×10⁹/L
   platelets <100×10⁹/L
   serum creatine >3.0× upper normal limit
   ALAT, ASAT >3.0× upper normal limit (5.0× in patients with liver metastases as the only likely cause of enzyme alteration)
   alkaline phosphatase >3.0× upper normal limit (5.0× in patients with liver or bone metastases as the only likely cause of enzyme alteration)
   bilirubin >3.0× upper normal limit
3. Participation in any investigational drug study within 4 weeks preceding treatment start.
4. Patients with clinically significant and uncontrolled renal- or hepatic disease.
5. Clinically significant cardiac disease: congestive heart failure (New York Heart Association class III or IV); symptomatic coronary artery disease; cardiac arrhythmia not well controlled with medication; myocardial infarction within the last 12 months.
6. Any serious underlying medical condition (at the judgement of the investigator) which could impair the ability of the patient to participate in the trial (e.g. active autoimmune disease, uncontrolled diabetes, etc.).
7. Any concomitant drugs contraindicated when administering Erbitux™ or Caelyx™ according to the Swissmedic-approved product information.
8. A cumulative doxorubicin dose of >300 mg/m² BSA (or cardiotoxic anthracycline-equivalent).
9. Patients with a history of uncontrolled seizures, central nervous system disorders or psychiatric disability judged by the investigator to be clinically significant and precluding informed consent or interfering with compliance.
10. Brain metastases.

### 2. Safety Parameters

### 2.1. Adverse Events (Primary Objective)

AU adverse events encountered during the clinical study will be recorded in the patients' history/file.

The intensity of clinical adverse events will be (waded according to the NCI CTC grading system Version 3.0 (http://ctep.info.nih.gov/reporting/ctc.html).

### 2.2. Laboratory Parameters

Prior to study onset, the normal values of the participating laboratories have to be recorded. The following laboratory procedures have to be carried out during the study:
every week (before new administration of study medications if appropriate):
   hemoglobin—leukocytes count including differential blood count
   platelet count
every 4 weeks (before new administration of study medications):
   ASAT-ALAT
   bilirubin
   alkaline phosphatase
   serum creatine
   LDH
   calcium
   urine analysis (“U-Status”: detection of erythro-, leuco- and proteinuria)
Pharmacokinetic study only during cylcle 1:
   Blood sample (2×7.5 ml serum tubes) at 0, 24, 48, 96 h and on day 8

### 2.3. Vital Signs and Physical Examination

The following vital signs and results of physical examination have to be documented prior to study start:
body temperature
blood pressure
heart rate
height (once at screening)
weight
performance status (ECOG)

### 2.4. Special Investigation

For pharmacokinetic studies, a blood sample (2×7.5 ml serum tubes) will be drawn at 0, 24, 48 and 96 hours as well as on day 8. Plasma will be separated from whole blood by centrifugation and frozen at −80° C. for further analysis. Doxorubicin concentration will be determined by fluorescence. Due to rapid clearance of free doxorubicin, this simple analysis provided an excellent measurement of circulating intact C225-ILs-dox. Pharmacokinetic parameters will be determined by noncompartmental pharmacokinetics data analysis using PK Solution 2.0 software (Summit Research Serviced, Montrose, Colo., USA).

### 2.5. Dose Modification for Toxicity

This is a dose escalation study (Phase I). For details see also section 10.3.2.

In an individual patient who experiences toxicity (DLT) but benefits from therapy, continuation of treatment at a reduced dose, determined according to the clinical judgment of the primary investigators, is an option (off study).

If possible, toxicities should be managed symptomatically. If toxicity occurs, the appropriate treatment will be used to ameliorate signs and symptoms including antiemetics for nausea and vomiting, antidiarrhoeals for diarrhoea, antipyretics and antihistamines for drug fever and 50% DMSO ointment for skin toxicity.

### 2.6. Supportive Measures

2.6.1. Nausea/Vomiting

A prophylactic antiemetic treatment should be given to the patients from the first cycle on. The use of a 5-HT3-receptor-antagonist is recommended. More aggressive antiemetic prophylaxis should be given to any patient who experiences grade ≧3 nausea/vomiting in a preceding cycle.

If, despite appropriate medication, grade ≧3 nausea/vomiting persists, the patient must be withdrawn from the study.

### 2.6.2. Diarrhea

No prophylactic anti-diarrhea treatment is recommended for the first cycle. However, following the first episode of diarrhea, the patient should receive symptomatic treatment with loperamide: 4 mg following the first episode, then 2 mg following each new episode until recovery of diarrhea (no more than 16 mg daily).

If, despite the appropriate medication, grade 3 diarrhea persists, the patient must be withdrawn from the study.

### 2.6.3. Palmer Plantar Erythema (PPE=Hand Foot Syndrome)

A prophylactic treatment should be given to the patients from the first cycle. The patient should receive 8 mg of dexamethason BID orally on days -1-4, 4 mg BID on day 5 and 4 mg on day 6. Additionally, patients should receive 150 mg pyridoxin (Vitamin B6) daily during the treatment period (orally) (20). If, despite the appropriate medication, grade 2 or 3 PPE occurs, administration of C225-ILs-dox should be interrupted for a maximum of 14 days. Once the PPE decreases in severity to CTC grade 1, the patient may continue treatment (if not defined as DLT).

If despite prophylactic and symptomatic treatment grade 2 or 3 toxicity remains, the patient must be withdrawn from the study.

### 3. Disease Evaluation (Efficacy Criteria)

### 3.1. Overall Response Rate (Secondary Objective)

Although response rate is not the primary endpoint of this trial, patients with measurable disease will be assessed by standard criteria. Tumor assessments will be done during screening and after 2, 4 and 6 cycles of treatment. After treatment completion, an assessment is performed every 3 months for the first year and then according to clinical needs. If progression is documented, no further assessments will have to be performed within the study. In responding patients, the response must be confirmed a minimum of 4 weeks after the response has first been recorded.

The primary efficacy criteria is the overall response rate which will be assessed according the RECIST criteria for reporting results of cancer treatment given in appendix 1.

Consistency of consecutive CT-scans and X-rays (e.g. the use of contrast etc.) must be ensured during all assessments for each patient with the same technique being used throughout the treatment period for evaluating the lesions.

### 3.2. Time to Progression

Time to progression will be measured from the time the patient has started treatment, to the time the patient is first recorded as having disease progression.

Disease progression must be adequately documented and assessed according to RECIST criteria.

### 4. Study Procedures

### 4.1. Screening

Informed consent must be given before any study specific screening procedures are performed.

The screening procedure may be done in two stages. The first group of assessments may be done at any time within 4 weeks prior to treatment start on day 1. The second group must be done within 7 days prior to treatment start. If the assessments are undertaken on day 1 they must be completed prior to study drug administration.

Assessments Day -28 to Day 1 (first day of C225-ILs-dox, prior to drug administration)

| Assessment | Includes |
|---|---|
| Patient's informed consent | Written consent |
| Demographic data | Date of birth |
| | Race |
| | Sex |
| History of malignant disease | Primary diagnosis |
| | Histology |
| | Location of metastases |
| Medical history | Concomitant non-malignant disease |
| | Treatment for non-malignant con- |
| | comitant disease (=concomitant |
| | medication) |
| General physical examination | Total body examination |
| Special examinations | ECG, Echocardiography |
| | Pregnancy test if requested |
| Tumor measurement/assessment | CT scan, MRI scan, ultrasound, or X- |
| | ray; clinical measurement in case of |
| | skin or palpable lymphnode metastases |
| Special examination | EGFR overexpression immunohisto- |
| | chemistry (DAKO) |

Assessments Day -7 to Day 1 (first day of C225-ILs-dox, prior to drug administration)

| Assessment | Includes |
|---|---|
| Vital signs and physical | Body temperature |
| measurements | Blood pressure |
| | Heart rate |
| | Height |
| | Weight |
| | Performance status (ECOG) |
| | Physical examination |
| General laboratory tests | Hematology |
| | Blood chemistry |

### 4.2. During Treatment

Tumor assessments will be done during screening and after 2, 4 and 6 cycles of treatment. After treatment completion, an assessment is performed every 3 months for the first year and thereafter according to clinical needs. If progression is documented, no further assessments will have to be performed within the study. In responding patients, the response must be confirmed a minimum of 4 weeks after the response has first been recorded.

Laboratory safety assessments:
Hemoglobin, leukocytes and thrombocytes will be analyzed weekly during the first cycle and every two weeks during subsequent cycles if not clinically indicated otherwise.
Transaminases, bilirubin, alkaline phosphate, creatinine, calcium, LDH and urine status will be analyzed every 4 weeks.

Adverse events will be recorded at each visit.

An echocardiography will be performed before, after 2 and 6 cycles of treatment (or at the end of study), and if clinically indicated in all patients.

### 5. Study Design

This is a single center, open study.

The recruitment of patients will be performed in two stages. First, patients will be enrolled according to section 10.3.2. (dose regimen and dose adjustment). The second stage allows an additional recruitment of up to 6 additional patients on the dose level defined as the MTD.

Patients having completed the treatment phase (24 weeks) and showing complete or partial response as well as stable disease will enter the observation phase of the study. This phase will end 12 months after the last patient has been included.

At any time during treatment phase or observation phase, patients with signs of disease progression according to RECIST criteria for reporting results of cancer treatment given in appendix 1 or having discontinued treatment due to unacceptable toxicity will go off study and be treated at the investigator's discretion.

### 6. Study Medication

### 6,1. Drug Names, Formulation, Storage

C225-IL-dox will be supplied for use as a solution of 10 mg doxorubicin per 20 ml vial for parenteral administration (0.5 mg doxorubicin/ml). C225-ILs-dox should be stored at 2-8° C.

### 6.1.1. Liposome Preparation

Liposomes were prepared by a lipid film hydration-extrusion method using repeated freeze-thawing to hydrate the lipid films (23). Liposomes were composed of 1,2-distearoyl-sn-glycero-3-phosphocholine (DSPC) and cholesterol (molar ratio 3:2) with methoxy polyethylene glycol (mPEG)-1,2-distearoyl-3-sn-glycerophosphoethanol-amine (DSPE; 0.5-5 mol % of phospholipid; Avanti Polar Lipids; Alabaster, Ala.). Following hydration, liposomes were extruded 10 times through polycarbonate filters (0.1 μm pore size). Liposome size was determined by dynamic light scattering (typically 80-100 nm). Phospholipid concentration was measured by phosphate assay (25).

For liposomes loaded with ADS645WS (American Dye Source, Quebec, Canada), the fluorescent dye (5 mmol/L) was dissolved in buffer for rehydration of the dried lipids. After passive loading, unencapsulated dye was removed using Sephadex G-75 chromatography.

For encapsulation of chemotherapeutic drugs doxorubicin (Bedford Laboratories, Bedford, Ohio) and epirubicin (Pharmacia, Kalamazoo, Mich.), a standard remote-loading method using ammonium sulphate was done (26, 27). For encapsulation of vinorelbine, liposomes were prepared as described following hydration in a solution of triethylammonium sucrose octasulfate (TEA₈SOS; 0.65 mol/LTEA, pH 5.2-5.5). Unentrapped TEA₈SOS was removed on a Sepharose CL-4B size exclusion column. Vinorelbine was added at a drug-to-phospholipid ratio of 350 g drug/mol phospholipid and the pH adjusted to 6.5 with 1 N HCl before initiation of loading at 60° C. for 30 minutes. The resulting liposomal vinorelbine was purified on a Sephadex G-75 column to remove unencapsulated drug.

### 6.1.2. Preparation of Monoclonal Antibody Fragments and Immunoliposomes

Intact 0225 mAb (cetuximab. Erbitux; ImClone Systems, in., New York, N.Y.) was cleaved and reduced as previously described (11). Fab′ fragments were covalently conjugated to maleimide groups at the termini of PEG-DSPE chains (Mal-PEG-DSPE; Nektar, Huntsville, Ala.; ref. 8). Conjugation efficiencies were typically 30% to 50% for C225-Fab′. For incorporation into preformed liposomes or commercial PLD (Doxil, Alza Pharmaceuticals, Palo Alto, Calif.), mAb conjugates were incorporated into liposomes by coincubation at 55° C. for 30 minutes at protein/liposome ratio of 30 μg Fab′/μmol phospholipid, resulting in incorporation efficiencies of 70% to 80% (11)

### 6.1.3. Formulation

C225-ILs-dox will be prepared in the pharmacy of the University Hospital of Basel (Prof. C. Surber). C225-ILs-dox will be stored in HEPES-Buffered-Saline (0.9% NaCl; HEPES 2 mM) at a pH of 6-7 in a concentration of 0.5 mg doxorubicin/ml. C225-ILs-dox will be added to 250 ml of 5% glucose for injection (500 ml for dose levels 50 mg/m2 and above). This formulation must be used within 24 hours after dilution in glucose. Diluted C225-ILs-dox should be a clear and reddish solution without any signs of aggregation.

### 6.1.4. Storage Requirement

Vials of C225-ILs-dox have to be stored in the refrigerator at a temperature ranging from 2°-8° C. to ensure optimal retention of physical and biochemical integrity. It is important not to freeze the study drug, since liposomes would be disrupted. C225-ILs-dox may be sensitive to shear-induced stress (e.g. agitation or rapid expulsion from a syringe). Vigorous handling (such as shaking) of C225-ILs-dox solution may results in aggregation of the protein and may create cloudy solutions. Vials are designed for single use only.

### 6.2 Packaging and Labeling

The vials of the study medication C225-ILs-dox are labeled as follows:
FOR CLINICAL TRIAL USE ONLY
Study CC1
C225-ILs-dox
Total content: 20 ml at 0.5 mg doxorubicin/ml=10 mg/vial
Store between 2-8° C. (DO NOT FREEZE)
Expiry date:
Batch ID:
Investigator name:
Patient identification:

### 6.3. Study Treatment

### 6.3.1. Rationale for Dose Selection

The standard dose of Caelyx used in numerous phase II and III trials and also in routine oncology practice is 40-50 mg/m2 given as a short infusion every 4 weeks. One of the main toxicities of the drug given at that dosage is palmar plantar erythema (PPE=hand foot syndrome). Similarly, an important possible side effect of Cetuximab is skin toxicity, usually manifesting itself as an acneiform rash of the face and trunk. This side effect is probably a consequence of the fact that the epidermis expresses EGFR at a relatively high level. Therefore, the main safety concern of this study is that directing Caelyx to EGFR-overexpessing cells via the anti-EGFR antibody Cetuximab might also increase the skin toxicity of the drug.

Treatment within this phase I study was at a very low dose of Caelyx, i.e. a 10^{th} of the standard dose of the drug (corresponding to an antibody (Cetuximab) dosage of approx. 0.9 mg/m2 compared to 250 mg/m2 (loading dose 400 mg/m2) in established clinical regimens), and to escalate dosage in small increments.

### 6.3.2. Dosage Regimen and Dose Adjustment

Patients will be treated in cohorts of three patients each at the following dose levels (quantification and dose levels of C225-ILs-dox are defined in mg doxorubicin):
Level 1=5 mg/m2
Level 2=10 mg/m2
Level 3=20 mg/m2
Level 4=30 mg/m2
Level 5=40 mg/m2
Level 6=50 mg/m2
Level 7=60 mg/m2
Level 8=70 mg/m2
Level 9=80 mg/m2

At each dose level, 3 patients may be enrolled simultaneously. Escalation to the next higher dose will be allowed after patient 3 of a given dose level has received at least one full cycle of therapy if no dose limiting toxicity (DLT) occurred at a given dose level. The decision to enter a next dose level will be made by a team after reviewing all available toxicity data of the previous groups. A DLT is defined as any grade 4 toxicity, any grade 3 toxicity lasting more than one week or/and febrile neutropenia grade 3 (defined as neutrophils <1.0×10e9/l and fever >38.5° C.). Nausea, vomiting, anorexia, and alopecia (grade 2) will be excluded as dose limiting toxicities. Similarly, adverse events that are clearly related to the primary tumor, such as progression of disease will not be considered as DLTs. In addition, preexisting toxicities must be taken into account when defining and analyzing DLTs.

Examples of grade 3 toxicities considered as DLT:
In the case of PPD (Hand Foot Syndrome), grade 3 toxicity is defined as ulcerative dermatitis or skin changes with pain interfering with function, and therefore considered as DLT.
In the case of diarrhea, grade 3 toxicity is defined as increase of >7 stools per day over baseline; incontinence; i.v. fluids >24 hrs and/or hospitalization, and therefore considered as DLT.
In the case of left ventricular function, grade 3 toxicity is defined as symptomatic cardiac dysfunction responsive to intervention and/or a decrease of the ejection fraction <40%, and therefore considered as DLT.

If a DLT occurs at any dose level, the following rules will apply:

| Number of Patients | |
|---|---|
| with DLT at a given | |
| Dose Level | Escalation Decision Rule |
| 0 out of 3 | Enter 3 patients at the next dose level |
| ≧2 | Dose escalation will be stopped. This dose |
| | level will be declared the maximally |
| | administered dose (highest dose |
| | administered). Three (3) additional patients |
| | will be entered at the next lowesr dose level if |
| | only 3 patients were treated previously at that |
| | dose. |
| 1 out of 3 | Enter at least 3 more patients at this dose |
| | level. |
| | If 0/3 or 1/3 of these 3 patients experience |
| | DLT, proceed to the next dose level. |
| | If 2/3 or more of this group suffer DLT, then |
| | dose escalation is stopped, and this dose is |
| | declared the maximally administered dose. |
| | Three (3) additional patients will be entered at |
| | the next lower dose level if only 3 patients |
| | were treated previously at that dose. |
| ≦2 out of 6 at | This is generally the recommended phase 2 |
| highest dose level | dose. At least 6 patients must be entered at |
| below the maximally | the recommended phase 2 dose. |
| administered dose | |

Sequential dose escalation will be allowed until a DLT is observed in 3/3-6 patients treated at the same dose level. At this point no further dose escalation will be allowed. The maximum tolerated dose (MTD) for potential future studies will than be defined as the dose level below the one at which the dose escalation had to be stopped.

In an individual patient who experiences toxicity, continuation of treatment at a reduced dose, determined according to the clinical judgment of the primary investigators and following the rules detailed in chapter 6.5, will be an option.

### 6.3.3. Treatment Duration

Patients will be treated until disease progression but for a maximum of 6 cycles.

### 6.4. Concomitant Treatment

All concomitant medication(s) must be reported in the case report form.

### 7. Premature Withdrawal

Patients may withdraw from the study at any time and for whatever reason, without affecting their right to appropriate treatment. The investigator has the right to withdraw a patient for any reason which is in the best interest of the patient, including intercurrent illness, adverse events, treatment failure or protocol violations.

A patient who becomes pregnant during the study will be withdrawn. The reason for drop out should be coded as protocol violation and pregnancy should be reported as Serious Adverse Event.

Although withdrawals should be avoided if at all possible, it is understood that withdrawals may occur during a study. Whenever a patient is withdrawn from a study, for whatever reason, a final study evaluation must be completed for that patient, staging the reason for withdrawal. All documentation concerning the patient must be as complete as possible.

### 8. Warnings and Precautions

Any adverse event that is considered SERIOUS must be reported IMMEDIATELY (within one working day) to Dr. Christoph Mamot or Prof. Christoph Rochlitz (both Division of Oncology, University Hospital of Basel; affiliation see title page of this protocol).

C225-ILs-dox therapy should only be initiated under supervision of a physician experienced in the treatment of cancer patients. Since this is a single center study performed at the Division of Oncology at the University Hospital in Basel only physicians of this division will perform the treatment in close collaboration with the investigators.

Regarding skin toxicity please also refer to 10.3.1 (rationale for dose selection).

### 9. Statistical Methods and Considerations

### 9.1. General Considerations

This is a phase study to evaluate the safety of C225-ILs-dox in patients with advanced solid tumors. Efficacy is a secondary endpoint of this study, therefore tumor measurements before, during and after therapy will provide some preliminary data also on tumor response to C225-ILs-dox. However, analysis of efficacy will be purely descriptive and no formal statistical tests will be performed.

### 9.2. Sample Size

The sample size for this trial is based on a study design used to provide a safety stopping rule in the event that dose-limiting toxicity (DLT) is encountered during the trial. The study plan is to enroll 3 patients at each dose level, with a maximum of another three additional patients to be entered sequentially at each of these dose levels depending on toxicity. The trial will be terminated when three out of three to six patients experience DLT at a particular dose level (DLT dose).

If the true toxicity rate at a dose level is “P”, then the probability of declaring the dose as toxic (DLT dose) is as follows:

| Toxicity Rate (P) | Probability of Detecting DLT Dose |
|---|---|
| 0.2 | 0.099 |
| 0.3 | 0.256 |
| 0.4 | 0.456 |
| 0.5 | 0.656 |
| 0.6 | 0.821 |
| 0.7 | 0.930 |
| 0.8 | 0.983 |

### 9.3. Primary and Secondary Analyses

### 9.3.1. Primary Variables

The adverse event profile of the patients for each dose level will be summarized in terms of frequency and number of events. Similarly, the number and proportion of patients who experience DLT will also be summarized. Listings of all adverse events and laboratory data will be provided.

### 9.3.2. Secondary Variables

The proportion of patients belonging to each of the response categories (see 7.2.1.) will be tabulated.

### 10. Ethical Considerations

This protocol has been written, and the trial is to be performed in accordance with the Declaration of Helsinki, the Guidelines of Good Clinical Practice issued by ICH and Swiss regulatory authorities' requirements.

Before entering any patients into this trial, the investigator has to make sure that the trial has been approved by the local ethics committee and that Swissmedic has opened the center.

### 10.1. Informed Consent and Patient Information

Informed consent shall be obtained on a written form approved by the local ethics committee and signed by the patient. Two informed consents have to be signed, one of which will be handed to the patient.

In seeking informed consent, the patient information provided in the appendix should be used (amended according to the requirements of the local ethics committee) and one copy should be handed to the patient.

The informed consent procedure must conform to the guidelines on Good Clinical Practice issued by ICH and Swissmedic.

All patients will be informed of the aims of the trial, the possible adverse experiences, how to react in case an adverse event occurs, and the procedures and possible hazards to which he/she will be exposed. They will be informed as to the strict confidentiality of their patient data, but they need to know that their medical records may be reviewed for trial purposes by authorized individuals other than their treating physician.

An investigator must provide the patient with sufficient opportunity to consider whether or not to participate and minimize the possibility of coercion or undue influence. The information provided shall be in a language intelligible to the patient and may not include any content that appears to waive any of the patient's legal rights, or appears to release the investigator, the sponsor, or the institution from liability for negligence.

It will be emphasized that participation is voluntary and that the patient is allowed to refuse further participation in the protocol whenever he/she wants. This will not prejudice the patient's subsequent care.

In case new data becomes available that shifts the risk/benefit ratio, the patient should reconsent.

11.

### 11. APPENDICES

### 11.1. Recist Criteria

Response Evaluation Criteria in Solid Tumors (RECIST) (29)

All patients with measurable disease will be evaluated for response.

Measurable disease—the presence of at least one measurable lesion. If the measurable disease is restricted to a solitary lesion, its neoplastic nature should be confirmed by cytology/histology.
Measurable lesions: lesions that can be accurately measured in at least one dimension with longest diameter³ 20 mm using conventional techniques or 10 mm with spiral CT scan.
Non-measurable lesions: all other lesions, including small lesions (longest diameter <20 mm with conventional techniques or <10 mm with spiral CT scan), i.e. bone lesions, leptomeningeal disease, ascites, pleural/pericardial effusion, inflammatory breast disease, lymphangitis cutis/pulmonis, cystic lesions, and also abdominal masses that are not confirmed and followed by imaging techniques.

Evaluation of Lesions

Evaluation of Target Lesions^{A} ^{A} All measurable lesions up to a maximum of 10 lesions representative of all involved organs should be identified as target lesions and recorded and measured at baseline. Target lesions should be selected on the basis of their size (lesions with the longest diameter) and their suitability for accurate repetitive measurements (either by imaging techniques or clinically). A sum of the long distance (LD) for all target lesions will be calculated and reported as the baseline sum LD. The baseline sum LD will be used as reference to further characterize the objective tumor response of the measurable dimension of the disease.
Complete Response (CR): Disappearance of all target lesions
Partial Response (PR): At least a 30% decrease in the sum of the longest diameter (LD) of target lesions taking as reference the baseline sum LD.
Progression (PD): At least a 20% increase in the sum of LD of target lesions taking as reference the smallest sum LD recorded since the treatment started or the appearance of one or more new lesions.
Stable Disease (SD): Neither sufficient shrinkage to qualify for PR nor sufficient increase to qualify for PD taking as references the smallest sum LD since the treatment started.

Evaluation of Non-Target Lesions^{B} ^{B} All other lesions (or sites of disease) should be identified as non-target lesions and should also be recorded at baseline. Measurements are not required, but the presence or absence of each should be noted throughout follow-up.
Complete Response (CR): Disappearance of all non-target lesions and normalization of tumor marker level,
Non-Complete Response: Persistence of one or more non-target lesions (non-CR) or/and maintenance of tumor marker level above the normal limits.
Progression (PD): Appearance of one or more new lesions and/or unequivocal progression of existing non-target lesions.^{C} ^{C} Although a clear progression of “non-target” lesions only is exceptional, in such circumstances, the opinion of the treating physician should prevail, and the progression status should be confirmed at a later time by the review panel (or trial chair).

Note:
Tumor markers alone cannot be used to assess response. If tumor markers are initially above the upper normal limit, they must normalize for a patient to be considered in complete clinical response when all lesions have disappeared.
Cytology and histology: If the measurable disease is restricted to a solitary lesion, its neoplastic nature should be confirmed by cytology/histology.

These techniques can be used to differentiate between PR and CR in rare cases (for example, residual lesions in tumor types such as germ cell tumors, where known residual benign tumors can remain).

The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

Evaluation of Best Overall Response

The best overall response is the best response recorded from the start of the treatment until disease progression/recurrence (taking as reference for progressive disease the smallest measurements recorded since the treatment started). The patients' best response assignment will depend on the achievement of both measurement and confirmation criteria.

| Target | Non-Target | New Lesions | |
|---|---|---|---|
| Lesions | Lesions | Response | Overall |
| CR | CR | No | CR |
| CR | Non-CR/Non-PD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Note:
Patients with a global deterioration of health status requiring discontinuation of treatment without objective evidence of disease progression at that time should be reported as “symptomatic deterioration”. Every effort should be made to document the objective progression even after discontinuation of treatment.
In some circumstances, it may be difficult to distinguish residual disease from normal tissue. When the evaluation of complete response depends upon this determination, it is recommended that the residual lesion be investigated (fine needle aspirate/biopsy) before confirming the complete response status.

Guidelines for Evaluation of Measurable Disease

All measurements should be recorded in metric notation using a ruler or calipers. All baseline evaluations should be performed within 14 days before registration according to the schedule of assessments.

Note: Tumor lesions in a previously irradiated area are not optimally considered measurable disease.

The same method of assessment and the same technique should be used to characterize each identified and reported lesion at baseline and during follow-up. CT and MRI are the best currently available and reproducible methods to measure target lesions selected for response assessment. Imaging-based evaluation is preferred to evaluation by clinical examination when both methods have been used to assess the antitumor effect of a treatment.
Clinical lesions will only be considered measurable when they are superficial (e.g. skin nodules, palpable lymph nodes). In the case of skin lesions, documentation by color photography including a ruler to estimate the size of the lesion is recommended.
Lesions on chest X-ray are acceptable as measurable lesions when they are clearly defined and surrounded by aerated lung. However, CT is preferable.
Conventional CT and MRI should be performed with cuts of 10 mm or less in slice thickness contiguously. Spiral CT should be performed using a 5 mm contiguous reconstruction algorithm. This applies to the chest, abdomen, and pelvis. Head & neck extremities usually require specific protocols.
When the primary endpoint of the trial is objective response evaluation, ultrasound (US) should not be used to measure tumor lesions that are clinically not easily accessible. It is a possible alternative to clinical measurements of superficial palpable nodes, subcutaneous lesions, and thyroid nodules. US might also be useful to confirm the complete disappearance of superficial lesions usually assessed by clinical examination.

Confirmatory Measurement/Duration of Response

Confirmation

In order to be assigned a status of PR or CR, changes in tumor measurements must be confirmed by repeat studies that should be performed 4 weeks after the criteria for response are first met. In the case of SD, follow-up measurements must have met the SD criteria at least once after trial at 7 weeks (see Schedule of assessments, appendix 23.18).

Duration of Overall Response

The duration of overall response is measured from the time measurement criteria are met for CR/PR (whichever is first recorded) until the first date that recurrent or progressive disease is objectively documented (taking as reference for progressive disease the smallest measurements recorded since the treatment started).

The duration of overall complete response is measured from the time measurement criteria are first met for CR until the first date that recurrent disease is objectively documented.

Duration of Stable Disease

Stable disease is measured from the start of treatment until the criteria for progression are met, taking as reference the smallest measurements recorded since the treatment started.

### 11.2. Eligibility Forms

Inclusion Criteria

Prior to enrollment in the study candidates must meet ALL the following criteria (check each box if OK):
1. Histologically proven locally advanced or metastatic solid tumor.
2. ECOG Performance 2.
3. No additional standard therapy available for the patient.
4. EGFR overexpression (according to DAKO EGFR pharmDx—Test) determined in the most recently evaluable tumor tissue.
5. No concomitant anti-tumor therapy (steroids are permitted—in breast cancer and prostate cancer, steroid dose needs to remain stable during the study period).
6. At least four weeks since termination of any previous anti-tumor treatment (6 weeks in the case of nitrosoureas or mitomycin C).
7. In patients with previous anthracycline exposure, a normal echocardiogram (LVEF >50%) is required.
8. Age ≧18.
9. Male or female.
10. Female and male patients of reproductive age must be using effective contraception.
11. Willing and able to sign an informed consent prior to participation in the study and to comply with the protocol for the duration of the study.

Exclusion Criteria

Candidates must be excluded from the study if ANY of the following criteria are met (check each box if OK):
1. Pregnancy and/or breastfeeding.
2. Patients with the following laboratory values
   neutrophils <1.5×10⁹/L
   platelets <100×10⁹/L
   serum creatine >3.0× upper normal limit
   ALAT, ASAT >3.0× upper normal limit (5.0× in patients with liver metastases as the only likely cause of enzyme alteration)
   alkaline phosphatase >3.0× upper normal limit (5.0× in patients with liver or bone metastases as the only likely cause of enzyme alteration)
   bilirubin >3.0× upper normal limit
3. Participation in any investigational drug study within 4 weeks preceding treatment start.
4. Patients with clinically significant and uncontrolled renal- or hepatic disease.
5. Clinically significant cardiac disease: congestive heart failure (New York Heart Association class III or IV); symptomatic coronary artery disease; cardiac arrhythmia not well controlled with medication; myocardial infarction within the last 12 months.
6. Any serious underlying medical condition (at the judgement of the investigator) which could impair the ability of the patient to participate in the trial (e.g. active autoimmune disease, uncontrolled diabetes, etc.).
7. Any concomitant drugs contraindicated when administering Brbitux™ or Caelyx™ according to the Swissmedic-approved product information.
8. A cumulative doxorubicin dose of >300 mg/m2 BSA (or cardiotoxic anthracycline-equivalent).
9. Patients with a history of uncontrolled seizures, central nervous system disorders or psychiatric disability judged by the investigator to be clinically significant and precluding informed consent or interfering with compliance.
10. Brain metastases.

### 12. Results

Preliminary results of the phase I trial are included in the following table. These results show that no or very little drug-related toxic effects can be observed up to a concentration of 50 mg/m². Particularly, no skin toxicity, particularly no palmar plantar erythema, was found at even the higher doses, while at the same time, clear signals of efficacy were observed, even at the lowest dose used.

TABLE 1
Preliminary results of the phase I trial

| | | | | | Efficacy |
|---|---|---|---|---|---|
| | Dose | | | Grade ¾ | (best |
| Pat. No. | (per m2) | Tumor | Cycles | Toxicity | response) |
| 1 | 5 mg | Prostate | 2 | none | PD* |
| 2 | 5 mg | Pancreatic | 2 | none | PD |
| 3 | 5 mg | Renal cell | 2 | none | PD |
| 4 | 10 mg | Pancreatic | 1 | none | n.e. |
| 5 | 10 mg | Esophageal | 3 | none | SD |
| 6 | 10 mg | Colorectal | 6 | none | SD |
| 7 | 20 mg | Colorectal | 2 | none | PD |
| 8 | 20 mg | Pancreatic | 1 | none | PD |
| 9 | 20 mg | Head and | 4 | none | SD |
| | | Neck | | | |
| 10 | 30 mg | Mesothelioma | 6 | none | PR |
| 11 | 30 mg | Prostate | 2 | none | PD |
| 12 | 30 mg | Pancreatic | 2 | none | PD |
| 13 | 40 mg | Bladder | 2 | none | PD |
| 14 | 40 mg | Bladder | 6 | none | SD (MR) |
| 15 | 40 mg | Renal cell | 1 | none | n.e. |
| 16 | 50 mg | Hepatocellular | 2 | Neutropenia | n.e. |
| | | | | Grade 3 | |

(*Minimal tumor progression after 2 cycles. Retrospectively, PSA decrease and remission of lung metastases for 18 months).
(no skin toxicity at all in all 16 patients treated so far)
PD Progression
SD Stable Disease
SD (MR) Stable Disease (Minimal Response)
PR Partial Response
n.e. not evaluated

### B Multi Drug Resistance Study Materials

### 1.1 Reagents

Reagents for liposome preparation included: DilC₁₈(3)-DS (Molecular Probes; Leiden, Netherlands); DSPC, cholesterol, and mPEG-DSPE (Avanti Polar Lipids; Alabaster, Ala., USA); Mal-PEG(2000/3400)-DSPE (Nektar; Huntsville, Ala., USA); organic solvents, and other chemicals of reagent purity (Sigma-Aldrich AG; Buchs, Switzerland).

Doxorubicin (Adriblastin RD®; Pfizer AG, Zürich, Switzerland) and pegylated liposomal doxorubicin (Caelyx®, Essex Chemie AG, Luzern, Switzerland) were obtained commercially from the pharmacy.

Immunoliposomes contained either Fab′ derived from C225 (cetuximab, Erbitux) or EMD72000 (matuzumab; both Merck KGaA, Darmstadt, Germany). Both monoclonal antibodies are recombinant IgG₁ that bind to the extracellular domain (ECD) of EGFR and thereby block activation by EGFR ligands such as EGF and TGF-α (36). While MAb C225 is a chimeric MAb, EMD72000 is a humanized MAb derived from transgenic mice (37).

MAb EMD72000 was kindly provided by Merck KGaA, Darmstadt, Germany,

### 1.2. Cell Lines

MDA-MB-231 human breast cancer and colorectal cancer cell lines HT-29 cancer cell lines were obtained from the department of research at the University of Basel or the American Type Culture Collection (ATCC). The resistant versions of theses cell lines were provided by Susan Bates (MDA-MB-231 Vb100; NIH, Bethesda, USA) and by Dr. Schäfer (HT-29 RDB: Charité, Berlin, Germany). MDA-MB-231 cells were maintained in “Improved MEM Zinc Option” medium (Invitrogen AG, Basel, Switzerland) and HT-29 in RPMI-1640 (Sigma-Aldrich AG, Buchs, Switzerland) supplemented with 10% fetal calf serum, 100 IU/ml penicillin and 100 μg/ml streptomycin in a humidified atmosphere of 95% air and 5% CO₂ at 37° C.

### 2. Liposome Preparation, Loading and Antibody Incorporation

### 2.1 Liposome Preparation

Unilamellar liposomes were prepared according to the repeated freeze-thawing method (23) using DSPC and Cholesterol (molar ratio 3:2) with mPEG-DSPE (0.5-5 mol % of phospholipid). Briefly, liposomes were subsequently extruded 10 times through polycarbonate filters with defined pore sizes of 0.1 μm, yielded liposomes of 90-120 nm diameter as determined by dynamic light scattering. Liposome concentration was measured utilizing a standard phosphate assay.

For uptake and internalization studies, liposomes were labeled with 0.1-0.3 mol % DilC₁₈(3)-DS, a fluorescent lipid that can be stably incorporated into liposomal membranes ((38) (39)).

For encapsulation of doxorubicin, the remote-loading method using ammonium sulfate was performed ((27)(26)). First, dry lipids were rehydrated in 250 mM ammonium sulfate at pH 5.5, followed by extrusion as described above. Free ammonium sulfate was removed by size-exclusion chromatography using a Sephadex G-75 column/HEPES buffered saline (pH 7.0). Liposomes were then incubated with doxorubicin for 30 min at 60° C. Under these conditions, loading efficiencies were typically in the range of 95-100% when 150 μg drug per μmol phospholipid was used. All unencapsulated doxorubicin was removed by size-exclusion chromatography using a Sephadex G-75 column. In addition, pegylated liposomal doxorubicin (PLD/Caelyx®/Doxil®) was obtained commercially.

### 2.2 MAb Fragment Preparation, Conjugation, and Liposome Incorporation

For C225- and EMD72000-Fab′, intact MAbs were incubated with pepsin (weight ratio 1:20) in 0.1 M sodium acetate (pH 3.7) at 37° C. for 3 h, followed by dialysis against HEPES-buffered saline (pH 6.0). The resulting F(ab)₂ were reduced with 2-mercapto-ethylamine or 2-mercaptoethanol under argon for 15 min at 37° C., and then recovered by gel filtration using Sephadex G-25. Reduction efficiency was typically 70-90%.

Fab′ were conjugated to Mal-PEG-DSPE as described previously ((11) (12)). Conjugation efficiencies were evaluated by SDS-PAGE, allowing comparison of free MAb fragment vs. conjugate; conjugation efficiencies were typically 30-50% for 0225 and 40-60% for EMD72000. For incorporation into preformed liposomes, including prepared liposomal drugs and probes or commercial pegylated liposomal doxorubicin, MAb fragment conjugates (Fab′-Mal-PEG-DSPE) which form micellar solutions, were incorporated into liposomes by coincubation at 55° C. for 30 min. As a result, the conjugates become attached to the outer lipid layer of the liposomes via hydrophobic DSPE domains. Unincorporated conjugates and free drug were separated from immunoliposomes by Sepharose CL-4B gel filtration. When DilC₁₈(3)-DS-labeled liposomes were used, <5% of the fluorescence was co-associated with the micelle fraction, indicating minimal transfer of this marker. Incorporation efficiency of conjugated MAb fragments was estimated by SDS-PAGE using a series of protein standards and gel scanning and quantitation as described. For both, 0225 and EMD72000, typically 75-85% of added MAb conjugate was incorporated into immunoliposomes, corresponding to 30-40 Fab′ fragments per liposome.

### 3. Study Designs

### 3.1 Binding and Internalisation Study

For flow cytometry studies, 250,000 cells were co-incubated in 12-well plates with saline (control), liposomes or EGFR-targeted immunoliposomes labeled with DilC₁₈(3)-DS for 2 h at 37° C., washed extensively with PBS, followed by detaching and storing on ice until subjected to flow cytometry. Fluorescence microscopy studies were performed accordingly except detaching the cells from the 12-well plates.

Immunoliposomes containing C225-Fab showed an approximately 2 orders-of-magnitude greater accumulation in the human breast cancer cell line MDA-MB-231 than did control liposomes, which produced only background levels of fluorescence in these cells. A similar pattern was found in the multi-drug resistant subcell line MDA-MB-231 Vb100.

Binding and uptake of C225-Fab′-containing immunoliposomes was also evaluated in EGFR-overexpressing colon cancer HT-29 cells and its multi-drug resistant subcell line HT-29 RDB. Here, immunoliposomes showed a more than 1 order-of-magnitude greater uptake in EGFR-overexpressing HT-29 cells, and comparable findings in the mdr cell line HT-29 RDB. In the non-EGFR overexpressing control cell line MCF-7 there was no difference in uptake/binding between non-targeted liposomes and anti-EGFR immunoliposomes (data not shown). These results indicate a high selectivity for immunoliposome uptake in both isogenic cell lines regardless of their mdr features.

The observation of minimal fluorescence uptake in target cells after incubation with control liposomes is consistent with the non-reactive properties of pegylated liposomes ((12) (40)). and also confirms that DilC₁₈(3)-DS can be used as a stable liposome-based marker without significant exchange into cell membranes.

### 3.2 Cytotoxicity Studies

Specific cytotoxicity of EGFR-targeted immunoliposomes containing doxorubicin was evaluated in target cells plated at a density of 8,000 cells per well in 96-well plates and allowed to grow overnight. Immunoliposomes or control treatments were applied for 2 h at 37° C., followed by washing with PBS and re-adding growth media. Cells were further incubated at 37 DC for 3 days and analyzed for cell viability using 3-(4,5-dimethylthiazol-2-yl)-2,5-diphenyl tetrazolium bromide (MTT) staining (41). For the cytotoxicity studies using the efflux pump inhibitor verapamil, this compound was added to the media at a concentration of 100 μM during the complete experiment.

In EGFR-overexpressing HT-29 wilde type colon cancer cells, EGFR-targeted immunoliposomal doxorubicin showed substantial in vitro cytotoxicity following treatment for 2 h (IC₅₀=0.25 μg/ml), which approached that of free doxorubicin (IC₅₀=0.3 μg/ml) (Table 2). Thus, EGFR-targeted immunoliposome delivery of doxorubicin was as efficient as the rapid diffusion of free doxorubicin, a small, amphipathic molecule that readily transverses cell membranes in vitro. On the other hand, EGFR-targeted immunoliposomal doxorubicin, derived by conjugation of C225-Fab′ to PLD, showed a much greater cytotoxicity than non-targeted PLD itself (IC₅₀ not reached) in HT-29 cells, indicating that delivery was antibody-dependent (Table 2). Notably, similar treatment with the antibody C225 alone for 2 h showed no cytoxicity in this assay, confirming that immunoliposome activity was due to targeted drug delivery and not related to potential antiproliferative effects of C225 during this brief incubation time. Furthermore, immunoliposomes containing C225-Fab′ but lacking encapsulated drug (“empty immunoliposomes”) similarly showed no cytotoxicity under these assay conditions. Also no effects of C225-immunoliposomes-dox have been seen in MCF-7 cells, which lack the EGF receptor (negative control; data not shown).

The identical experiment was performed in the multi-drug resistant sub cell line HT-29 ROB. Notably, in this cell line immunoliposomal delivery of doxorubicin (IC₅₀ 0.5 μg/ml) was superior to that of the free drug (IC₅₀=9.5 μg/ml; =19-fold) and also liposomal drug (IC₅₀ not reached). To sum up this part of our studies, while free doxorubicin was much less cytotoxic in the multi-drug resistant variant of the HT-29 cell line compared to the wild type, there was almost no difference for the immunoliposomal compound regardless of different mdr features in this cell lines, indicating that immunoliposomes are able to bypass multi-drug resistance mechanisms in this setting.

Immunoliposome-mediated cytotoxicity with doxorubicin was also evaluated in EGFR-overexpressing human breast cancer cell line MDA-MB-231 V13100 featuring multi-drug resistance and compared to results with its parental cell line MDA-MB-231 lacking mdr. In the parental wild-type MDA-MB-231, ILs containing C225-Fab′ were as efficient in delivering doxorubicin as free doxorubicin itself, which again can easily penetrate the cell membrane, and clearly more cytotoxic than non-targeted liposomal doxorubicin/PLD (IC₅₀=0.3 vs. 0.6 vs. 120 μg/ml.

Interestingly, in the highly drug resistant MDA-MB-231 Vb 100 cell line, ILs loaded with doxorubicin (dox) produced a 216-fold greater cytotoxicity than free dox, and were also markedly more cytotoxic than the non-targeted liposomal doxorubicin (IC₅₀=0.6 vs. 130 vs. >900 μg/ml.

The same experiment in resistant MDA-MB-231 Vb100 cells was repeated in the presence of verapamil. This substance is able to inhibit efflux pumps and therefore reverse specific mechanisms of multi-drug resistance. In fact, by adding verapamil to this experiment the multi-drug mechanism could be converted effectively and as a result free doxorubicin was as efficient as in the wild-type MDA-MB-231 cell line (IC₅₀=0.9 μg/ml). In contrast, the addition of verapamil did not further increase the efficacy of doxorubicin delivered by anti-EGFR immunoliposomes (IC₅₀=0.5 μg/ml), thus confirming our finding that ILs are able to overcome multi-drug mechanisms and that this delivery system is unaffected by the presence of efflux pumps.

TABLE 2
Summary Results of Cytotoxicity Study

| | HT-29 | | | |
|---|---|---|---|---|
| | WT | C225-ILs-dox | HT-29 RDB | C225-ILs-dox |
| | (IC₅₀) | vs. free dox | (IC₅₀) | vs. free dox |
| free dox | 0.3 | 1.2 - fold | 9.5 | 19 - fold |
| C225-ILs-dox | 0.25 | | 0.5 | |
| PLD | >31 | | >31 | |

PLD non-targeted liposomal doxorubicin
C225-ILs-dox C225 antibody targeted liposomal doxorubicin

### 3.3 Accumulation of Doxorubicin in the Cytoplasma and Nuclei

For comparative accumulation studies, tumor cells (HT-29, HT-29 ROB, MDA-MB-231 or MDA-MB-231 Vb100) have been plated at a density of 200,000 cells per well in 12-well plates. Free doxorubicin, non-targeted liposomal doxorubicin (PLD) and immunoliposomal doxorubicin have been applied at a doxorubicin concentration of 3 μg/ml for 2 h at 37° C., followed by 2 washing rounds with media. Verapamil was added to the experiment in a concentration of 0, 10 or 100 μM. After another 2 h incubation without any treatment cells were analyzed as follows:

After removing the media, cells were washed once with 1 ml of culture medium containing FCS, followed by 1 ml of PBS with calcium and magnesium at room temperature (RT) for 3 min. The PBS was replaced by 400 μl C100T solution (100 ml containing 2.1 g citric acid and 0.5 ml Tween 20. Shaking for 10-15 min at 300 cycles/min resulted in solubilization of the cell membrane and released the nuclei, as confirmed by microscopy. The complete content of each well was transferred to transparent 0.5 ml PCR tubes and centrifuged at RT and 1200 rcf for 5 min. This way, the nuclei will sediment at the tips of the tubes, which is crucial for further processing. For the determination of doxorubicin in the cytaplasma, 350 μl from the supernatant were removed and mixed with 350 μl acid methanol (methanol containing 1 M orthophosphoric acid). For nuclear accumulation of doxorubicin the pellet with the nuclei was washed twice with 500 μl PBS containing 1% C100T and using subsequent centrifugation as described before. After careful removing of the final supernatant, doxorubicin from the pellets was extracted overnight by 400 μl 50% acid methanol.

From both cytoplasmic and nuclear extracts, 300 μl were transferred into a 96-well plate and measured by a “SpertraMax Gemini Fluorimeter” (Molecular Devices).

### 3.4 Tumor Xenograft Models

Efficacy for non-targeted liposomes versus anti-EGFR immunoliposomes were studied in the MDA-MB-231 wild type and resistant breast cancer xenograft tumor model. Swiss nu/nu mice (5-6 weeks; Charles River, France) were injected subcutaneously (s.c.) with EGFR-overexpressing MDA-MB-231 tumor cells (1×10⁷ cells, wild type or resistant) into the back of the animal. Once tumor xenografts had become established and tumors measured 150-250 mm³, mice were randomly assigned to different treatment groups (8-10 animals/group, depending on study). All i.v. treatments were performed via tail vein injection, typically in 100-200 μl volume. Liposomes and anti-EGFR immunoliposomes (C225- and EMD72000-) were administered intravenously at a dose of 10 mg doxorubicin/kg/dose once weekly for 3 weeks, for a total dose of 30 mg dox/kg. Free drug was injected on the same schedule as liposomes or immunoliposomes intravenously at their MTD of 30 mg dox/kg for doxorubicin. In control groups, saline was administered intravenously at the same injection volume and schedule.

Tumor growth was monitored for a period of 55-100 days post tumor implantation. Mice were weighted and examined for toxicity three times a week. Tumor measurements were performed 2-3 times weekly using a caliper, and tumor volumes were calculated using the equation: (length × width²)/2.

In the wild-type MDA-MB-231 xenograft model lacking mdr features, anti-EGFR immunoliposome-dox was administered i.v. at a total dose of 30 mg dox/kg divided into three weekly doses of 10 mg/kg. Anti-EGFR immunoliposomes were either prepared from the anti-EGFR MAb C225 or from EMD72000. Control treatments included: saline; free doxorubicin and non-targeted liposomal doxorubicin (commercial pegylated liposomal doxorubicin; PLD) at the same dose and schedule as immunoliposomes.

Free doxorubicin produced some tumor growth inhibition when compared to saline treatment. Non-targeted liposome delivery of doxorubicin via PLD at this high dose induced tumor regression and clearly increased efficacy over free drug. Treatment with anti-EGFR immunoliposome-dox, regardless if C225 or EMD72000 was used, produced substantial tumor regressions and was overall the most efficacious treatment. Until day 77, tumor regressions were similar for the PLD, C225-ILs-dox and EMD72000-ILs-dox groups. However, during follow-up, tumors treated with untargeted PLD all started to regrow while tumors treated with immunoliposomal doxorubicin, both C225-ILs-dox and EMD72000-ILs-dox, did not show growth activity until the end of observation (day 100), suggesting even a curative potential of anti-EGFR immunoliposomes as previously reported in other xenograft tumor models.

The same experiment was repeated in the MDA-MB-231 Vb100 xenograft model featuring a very similar EGFR overexpression (data not shown) but additionally multi-drug resistance. Again anti-EGFR immunoliposome-dox derived either from C225 or EMD72000 were administered i.v. at a total dose of 30 mg dox/kg divided into three weekly doses of 10 mg/kg. Comparators included saline, free doxorubicin and non-targeted liposomal doxorubicin (commercial pegylated liposomal doxorubicin; PLD) at the same dose and schedule as immunoliposomes.

In this highly multi-drug resistant model, free doxorubicin did not show any tumor growth inhibition when compared to saline treatment. Non-targeted liposome delivery of doxorubicin via PLD at this high dose demonstrated some tumor growth inhibition. Interestingly and importantly, treatment with anti-EGFR immunoliposome-dox, regardless if C225 or EMD72000 was used, produced substantial tumor regressions and was overall the most efficacious treatment. C225-ILs-dox seemed to be moderately more officious compared to EMD72000-ILs-dox. However, this was only a trend and statistically not significant. Overall, the results of this experiment demonstrate that anti-EGFR immunoliposomes are effective even against multi-drug resistant tumors and can overcome mdr mechanisms. (see Table 3)

In both models, anti-EGFR immunoliposome-dox were well-tolerated by the mice. Treatment with anti-EGFR immunoliposome-dox was associated with no major weight loss:

TABLE 3
Results of Tumor Xenograft Study

| | | | MDA-231 Vb100 |
|---|---|---|---|
| IC 50 (ug/ml) | MDA-231 WT | MDA-231 Vb100 | verapamil |
| PLD | 120 | >900 | 740 |
| free dox | 0.6 | 130 | 0.9 |
| C225-ILs-dox | 0.3 | 0.6 | 0.5 |

### 3.5 Statistical Analysis

To evaluate the statistical significance of the results, tumor volumes were analyzed and different treatment groups were compared using Student's t-test (2-sample individual t-test) for each time point. In addition, a multivariate (rank) test was performed based on the sums of ranks for each mouse. Tumor size at each time point after last treatment was ranked across all mice for that day and the ranks were summed. The sum of the ranks was compared in each case for two treatments by a 2-sample t-test (42).

### REFERENCES

(1) Sridhar S S, Seymour L, Shepherd F A. Inhibitors of epidermal-growth-factor receptors: a review of clinical research with a focus on non-small-cell lung cancer. Lancet Oncol 2003; 4:397-406.
(2) Longley D B, McDermott U, Johnston P G. Predictive markers for colorectal cancer: current status and future prospects. Clin Colorectal Cancer 2003; 2:223-30.
(3) Baselga J, Pfister D, Cooper M R, Cohen R, Burtness B, Bos M, et al. Phase I studies of anti-epidermal growth factor receptor chimeric antibody C225 alone and in combination with cisplatin. J Clin Oncol 2000; 18:904-14.
(4) O'Brien M E, Wigler N, Inbar M, Rosso R, Grischke E, Santoro A, et al. Reduced cardiotoxicity and comparable efficacy in a phase III trial of pegylated liposomal doxorubicin HCl (CAELYX/Doxil) versus conventional doxorubicin for first-line treatment of metastatic breast cancer. Ann Oncol 2004; 15:440-9.
(5) Keller A M, Mennel R G, Georgoulias V A, Nabholtz J M, Erazo A, Lluch A, et al. Randomized phase III trial of pegylated liposomal doxorubicin versus vinorelbine or mitomycin C plus vinblastine in women with taxane-refractory advanced breast cancer. J Clin (Oncol 2004; 22:3893-901.
(6) Northfelt D W, Dezube B J, Thommes J A, Levine R, Von Roenn J H, Dosik G M, et al. Efficacy of pegylated-liposomal doxorubicin in the treatment of AIDS-related Kaposi's sarcoma after failure of standard chemotherapy. J Clin Oncol 1997;15:653-9.
(7) Stewart S, Jablonowski H, Goebel F D, Arasteh K, Spittle M, Rios A, et al. Randomized comparative trial of pegylated liposomal doxorubicin versus bleomycin and vincristine in the treatment of AIDS-related Kaposi's sarcoma. International Pegylated Liposomal Doxorubicin Study Group. J Clin Oncol 1998; 16:683-91.
(8) Gordon A N, Fleagle J T, Guthrie D, Parkin D E, Gore M E, Lacave A J. Recurrent epithelial ovarian carcinoma: a randomized phase III study of pegylated liposomal doxorubicin versus topotecan. J Clin Oncol 2001; 19:3312-22,
(9) Orditura M, Quaglia F, Morgillo F, Martinelli E, Lieto E, De Rosa G, et al. Pegylated liposomal doxorubicin: pharmacologic and clinical evidence of potent antitumor activity with reduced anthracycline-induced cardiotoxicity (review). Oncol Rep 2004; 12:549-56.
(10) Kirpotin D B, Hong K, Park J W, Shalaby R, Shao Y, Zheng W, et al, Anti-HER2 immunoliposomes produced by spontaneous capture of an amphipathic poly(ethylene glycol)-anti-HER2 atibody conjugate into the liposome membrane. Proc. Amer. Assoc, Cancer Res. 2000; 41:325.
(11) Mamot C, Drummond D C, Greiser U, Hong K, Kirpotin D B, Marks J D, et al. Epidermal growth factor receptor (EGFR)-targeted immunoliposomes mediate specific and efficient drug delivery to EGFR- and EGFRvIII-overexpressing tumor cells. Cancer Res 2003b; 63:3154-61,
(12) Kirpotin D, Park J W, Hong K, Zalipsky 5, Li W L, Carter P. et al. Sterically stabilized anti-HER2 immunoliposomes: design and targeting to human breast cancer cells in vitro. Biochemistry 1997; 36: 66-75.
(13) Mamot C, Drummond D C, Noble C, Kirpotin D B, Hong K, Park J W. EGFR/EGFRvIII targeted immunoliposomes significantly enhance the efficacy of multiple anticancer drugs in vivo. Cancer Res 2005.
(14) Khaw, B. A. et al. J. Nucl. Med. 23:1011-1019 (1982)
(15) Rousseaux et al. Methods Enzymology, 121:663-69, Academic Press, 1986
(16) Rader et al., Proc. Natl. Acad. Sci. USA (1998) 95:8910-8915
(17) Queen et al., Proc. Natl. Aced Sci LISA, 86:10029-10032 (1989), Hodgson at al., Bio/Technoloy, 9:421 (1991))
(17) Allen, T. M., et al., Biochemicia et Biophysica Acta, 1237:99-108 (1995)
(19) Zalipsky, S., Bioconjugate Chem., 4(4):296-299 (1993)
(20) Zalipsky, S., et al. FEBS Lett., 353:71-74 (1994)
(21) Zalipsky, S. et al., Bioconjugate Chemistry, 6(6):705-708 (1995)
(22) Zalipsky, S., in STEALTH LIPOSOMES (D. Lasic and F. Martin, Eds.) Chapter 9, CRC Press, Boca Raton, Fla. (1995)
(23) Szoka, F., Jr., at al., Ann. Rev, Biophys. Bioeng., 9:467 (1980)
(24) Martin, F. J., in SPECIALIZED DRUG DELIVERY SYSTEMS MANUFACTURING AND PRODUCTION TECHNOLOGY, P. Tyle, Ed., Marcel Dekker, New York, pp. 267-316 (1990)
(25) Bartlett G K, J Bio Chem 1959, 234: 466-8
(26) Lasic D D et al, FEBS Lett 1992, 312: 255-8
(27) Haran G et al, Biochim Biophys Acta 1993, 1151; 201-15
(28) Nellis D F et al, Biotech Prog 2005, 21: 221-32
(29) Therasse P, Arbuck S G, Eisenhauer E A, Wanders J, Kaplan R S, Rubinstein L, et al. New guidelines to evaluate the response to treatment in solid tumors. European Organization for Research and Treatment of Cancer, National Cancer Institute of the United States, National Cancer Institute of Canada. J Natl Cancer Inst 2000; 92:205-16.
(30) Kellen, J. A. (1994). The Phenomenon of multi drug resistance. In Kellen J A (ed) Reversal of MultiDrug Resistance in Cancer, CRC Press, Boca Raton, pp. 1-21.
(31) Mickley, L. & Fojo, A. T. (1998). The MDR genes. In: Pinedo H M, Giaccone G (eds.) Drug Resistance in the Treatment of Cancer, Cambridge University Press, Cambridge, pp. 101-131.
(32) Cole, S. P. & Deeley, R. G. (1998). Multidrug resistance mediated by the ATP-binding cassette transporter protein MRP. Bioessays, 20, 931-40.
(33) Juranka, P. F., Zastawny, R. L. & Ling, V. (1989). P-glycoprotein: multidrug-resistance and a superfamily of membrane-associated transport proteins. Faseb J, 3, 2583-92.
(34) Renes, J., de Vries, E. G., Jansen, P. L. & Muller, M. (2000). The (patho)physiological functions of the MRP family. Drug Resist Updat, 3, 289-302.
(35) Leonard, G. D., Polgar, O. & Bates, S. E. (2002). ABC transporters and inhibitors: new targets, new agents. Curr Opin Investig Drugs, 3, 1652-9.
(36) Fan, Z., Masui, H., Altas, I. & Mendelsohn, J. (1993). Blockade of epidermal growth factor receptor function by bivalent and monovalent fragments of 225 anti-epidermal growth factor receptor monoclonal antibodies. Cancer Res, 53, 4322-8.
(37) Bier, H, Hoffmann, T., Hauser, U., Wink, M., Ochler, M., Kovar, A., Muser, M. & Knecht, R. (2001). Clinical trial with escalating doses of the antiepidermal growth factor receptor humanized monoclonal antibody EMD 72 000 in patients with advanced squamous cell carcinoma of the larynx and hypopharynx. Cancer Chemother Pharmacol, 47, 519-24.
(38) Claassen, E. (1992). Post-formation fluorescent labelling of liposomal membranes. In vivo detection, localisation and kinetics. J immunol Methods, 147, 231-40
(39) Litzinger, D. C., Buffing, A. M., van Rooijen, N. & Huang, L. (1994). Effect of liposome size on the circulation time and intraorgan distribution of amphipathic poly(ethylene glycol)-containing liposomes. Biochim Biophys Acta, 1190, 99-107
(40) Park, J. W., Hong, K., Carter, P., Asgari, H., Guo, L. Y., Keller, G. A., Wirth, C., Shalaby, R., Kotts, C., Wood, W. I. & et al. (1995). Development of anti-p185HER2 immunoliposomes for cancer therapy. Proc Natl Acad Sci U S A, 92, 1327-31.
(41) Scudiero, D. A., Shoemaker, R. H., Paull, K. D., Monks, A., Tierney, S., Nofziger, T. H., Currens, M. J., Seniff, D. & Boyd, M. R. (1988). Evaluation of a soluble tetrazolium/formazan assay for cell growth and drug sensitivity in culture using human and other tumor cell lines. Cancer Res, 48, 4827-33.
(42) O'Brien, P. C. (1984). Procedures for comparing samples with multiple endpoints. Biometrics, 40, 1079-87.

U.S. Pat. Nos. 5,843,708; 6,180,370; 5,693,762; 5,585,089; 5,530,101, 5,565,332; 5,639,641, 5,395,619, 5,013,556, 5,631,018. and WO 98/07409

1. An immunoliposome comprising an antibody or an antibody fragment, which recognizes and binds to an EGF receptor antigen on the surface of a solid tumor and further encapsulating in the liposome an anti-tumor compound, for multi-line treatment of cancer.

2. An immunoliposome according to claim 1, wherein said tumor is an EGFR-positive tumor.

3. An immunoliposome according to claim 1, wherein the liposome encapsulates a cytostatic compound.

4. An immunoliposome according to claim 1, wherein the antitumor compound is a compound selected from the group consisting of daunomycin, idarubicin, mitoxantrone, mitomycin, a platinum analog, vincristine, epirubicin, aclacinomycin, methotrexate, etoposide, doxorubicin, cytosine arabinoside, a fluorinated pyrimidine, purine, or nucleoside, thiotepa, BCNU, a taxane derivative or isolate, camptothecins, polypeptides, and a nucleic acid.

5. An immunoliposome according to claim 4, wherein the cytotoxic compound is a compound selected from the group consisting of doxorubicin, epirubicin and vinorelbine.

6-17. (canceled)

18. A method according to claim 39, wherein the amount of immunoliposome or a pharmaceutical composition in the treatment is such as to exhibit no or substantially no toxic side effects.

19. A method according to claim 39, wherein the treatment is topical and does not show skin toxicity.

20. A method according to claim 39, wherein the treatment does not cause the patient to show palmar plantar erythema.

21. A method according to claim 39, wherein the treatment is topical and shows no or substantially no toxic side effects at a concentration of between 5 mg/m² and 80 mg/m².

22. An immunoliposome according to claim 21, wherein the treatment shows no or substantiality no toxic side effects at a concentration of up to 40 mg/m².

23. An immunoliposome according to claim 1, wherein the antibody or antibody fragment is covalently bound to the liposome membrane.

24. An immunoliposome according to claim 1, wherein the antibody is covalently conjugated to the terminus of a linker molecule anchored to the liposome.

25. An immunoliposome according to claim 24, wherein the linker molecule is a polyethylene glycol.

26. An immunoliposome according to claim 1, wherein the antibody is a monoclonal antibody directed to the ligand-binding extracellular domain of the EGF receptor.

27. A method according to claim 39, wherein the cancer is selected from the group consisting of Kaposi's sarcoma, recurrent ovarian cancer, soft tissue sarcoma, glioma, melanoma, mesothelioma, transitional cell carcinoma of the urothelial tract, endometrial, pancreatic, small-cell and non-small-cell lung, hepatocellular, renal cell, esophageal, colorectal, anal, vaginal, vulvar, prostate, basal cell carcinoma of the skin head and neck, and choiangio carcinoma.

28. A method according to claim 39, wherein the cancer is selected from the group consisting of prostate, pancreatic, kidney, urothelial, oesophageal, head and neck, colonrectal, a hepatocellular cancer and a mesothelioma.

29. A method according to claim 39, wherein said patient is suffering from a prostate cancer with a tumor that has progressed on hormonal and/or docetaxel and/or mitoxanthrone treatment.

30. A method according to claim 39, wherein said patient is suffering from a pancreatic cancer with a tumor that has progressed on gemcitabine and/or capecitabine and/or oxaliplatin treatment.

31. A method according to claim 39, wherein said patient is suffering from a kidney cancer with a tumor that has progressed on interferon and/or capecitabine and/or sunitinib and/or sorafinib treatment.

32. A method according to claim 39, wherein said patient is suffering from an esophageal cancer with a tumor that has progressed on cisplatinum and/or 5-FU and/or docetaxel and/or cetuximab treatment.

33. A method according to claim 39, wherein said patient is suffering from a colon and/or rectal cancer with a tumor that has progressed on cetuximab and/or Bevacizumab and/or oxaliplatin and/or irinotecan and/or capecitabine and/or 5-FU treatment.

34. A method according to claim 39, wherein said patient is suffering from a urothelial cancer with a tumor that has progressed on cis- or carboplatinum and/or gemcitabine and/or doxorubicin and/or methotrexate and/or vincristin.

35. A method according to claim 39, wherein said patient is suffering from a mesothelioma with a tumor that has progressed on cis- or carboplatinum and/or gemcitabine and/or pemetrexed.

36. A method according to claim 39, wherein said patient is suffering from a hepatocellular cancer with a tumor that has progressed on sunitinib and/or sorafenib.

37. A method according to claim 39, wherein the cancer represented by is a locally advanced or metastatic tumor, wherein a response rate is achieved of between 5% and 95%.

38. A pharmaceutical composition comprising an immunoliposome according to claim 1 together with a pharmaceutically acceptable carrier or excipient or a diluent for first- to multi-line, particularly for second-line, particularly third-line, particularly fourth-line, particularly fifth-line, particularly sixth-line, particularly seventh- and higher-line treatment of cancer, particularly a cancer represented by a locally advanced or metastatic tumor, particularly an EGFR-positive tumor, in a human patient in a clinical set-up.

39. A method of treatment of cancer in a human patient comprising administering to said human patient an immunoliposome or a pharmaceutical composition according to claim 1.

40. A method according to claim 39, wherein the cancer is a locally advanced or metastatic tumor, and the patient has received, but not responded to, at least one other cancer treatment noliposome or a pharmaceutical composition according to any of the preceding claims.

41. A method according to claim 39, wherein the cancer is an EGFR-positive tumor, in a human patient in a clinical set up.

42. A method according to claim 40, wherein the patient has received, to at least two other cancer.

43. A method according to claim 39, wherein the patient is multi-drug resistant.

44. (canceled)

45. A method according to claim 39, wherein the cancer is breast cancer.

46. A method according to claim 39, wherein the cancer is colonrectal cancer.

47-48. (canceled)

49. A pharmaceutical composition comprising an immunoliposome according to claim 38, wherein said immunoliposome encapsulates doxorubicin and further comprises antibody MAb C225 or antibody EMD72000 or a fragment thereof, which still exhibits the specific binding properties of one or both of said ant
